(19) United States
(12) Patent Application Publication
(10) Pub. No.: US 2016/0051590 A1
(43) Pub. Date: Feb. 25, 2016

(54) COMPOSITIONS AND METHODS FOR TREATMENT OF VESSEL DISEASE

(71) Applicant: UNIVERSITY OF LOUISVILLE RESEARCH FOUNDATION, INC, Louisville, KY (US)

(72) Inventors: James Hoying, Louisville, KY (US); Marvin E. Morris, Prospect, KY (US); Stuart K. Williams, Harrods Creek, KY (US)

(21) Appl. No.: 14/888,491

(22) PCT Filed: May 1, 2014
(86) PCT No.: PCT/US14/36371

§ 371 Date: Nov. 2, 2015

Related U.S. Application Data
(60) Provisional application No. 61/818,294, filed on May 1, 2013.

(51) Int. Cl. A61K 35/35; C12N 5/077; A61K 9/00

(52) U.S. Cl. CPC A61K 35/35; A61K 9/0019; C12N 5/0652

(57) ABSTRACT

The present disclosure relates to compositions and methods for the treatment of blood vessel disease. More specifically, the compositions and methods of the present disclosure make use of a population of stromal vascular fraction cells. In some embodiments, the population of stromal vascular fraction cells comprises at least one macrophage.

### RELATED APPLICATION

This application claims priority from U.S. Provisional Patent Application Ser. No. 61/818,294, filed on May 1, 2013, the entire disclosure of which is incorporated herein by this reference.

### TECHNICAL FIELD

The presently-disclosed subject matter relates to compositions and methods for the treatment of blood vessel disease.

### BACKGROUND

Blood vessel disease commonly refers to any disease or disorder in which blood vessel dysfunction or blood vessel narrowing leads to tissue damage and cell death as a result of less oxygen-rich blood being supplied to particular tissues or cells. Such narrowing of blood vessels can arise as a result of plaque build-up on the walls of the vessels or chronic inflammation, and can include conditions such as ischemic disease (peripheral artery disease, angina, heart attack, stroke, etc.), Reynaud's disease, Brueger's disease, hypertension, chemotherapeutic compromise, and erectile dysfunction. In each condition, however, the blood vessel disease frequently results in distal vessel injury and/ or dysfunction that, in turn, complicates and, in many cases, exacerbates revascularization strategies and recovery from ischemic injury.

Indeed, small vessel dysfunction causes and/or complicates an array of disease conditions including systemic vascular disease (Wu, et al., 2013), central and peripheral ischemia (Olsen, et al, 1983; Jeremy, et al., 1987; Faber, et al., 2011; Bauer, et al., 2005), diabetes (Georgi, et al, 2011, Duncan, et al, 1986), cancer (Pries, et al., 2010; Pries, et al., 2009), and hypertension (Humar, et al, 2009; Mourad, et al., 2011).

Moreover, dysregulation of the small arteries and arterioles compromises end tissue/organ function due to an inability to (i) maintain proper flow reserve, (ii) establish normal baseline flow resistance, and/or (iii) preserve dynamic blood-tissue interactions. This distal vascular dysfunction arises via a variety of mechanisms negatively impacting endothelial and/or mural cell activities and often involves numerous individual vessels across an entire vascular bed. Meanwhile, there are relatively few therapeutic options for treating small vessel disease, due in part to the diffuse and complex nature of the problems. Accordingly, there remains a need in the art for additional compositions and methods useful in the treatment of blood vessel disease.

### BRIEF SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of features.

In some embodiments, the present disclosure provides a method of treating a blood vessel disease, wherein the method comprises at least the step of administering to a subject a composition comprising a stromal vascular fraction cell population. The method may comprise the step of intravenously injecting the composition. Further, in some embodiments, the method may include the step of isolating the stromal vascular fraction cell population from adipose tissue of the subject prior to administering the composition to the subject. And in certain embodiments, administering the stromal fraction cell population to the subject comprises distributing the stromal vascular fraction cell population in at least one of the intima, media, and adventitia of a blood vessel of the subject.

The present disclosure provides that, in some embodiments, administering the stromal vascular fraction cell population to a subject increases an amount of a vasodilatory agent in the subject. For example, in certain embodiments, the vasodilatory agent is chosen from nitric oxide, histamine, prostacyclin, prostaglandin E2, prostaglandin I2, leukotriene C4, leukotriene D4, leukotriene E4, vasoactive intestinal peptide (VIP), adenosine, adenosine triphosphate, adenosine diphosphate, L-arginine, bradykinin, substance P, nicotinic acid, platelet activating factor, carbon dioxide, lactic acid, natriuretic peptide, heparin, heparin sulfate, and endothelium derived hyperpolarizing factor.

In certain embodiments, the step of administering the composition to the subject decreases an amount of vasoconstriction in a blood vessel of the subject. And in some embodiments, the step of administering the composition decreases the activity of a vasoconstricting agent in a blood vessel of a subject. Moreover, the present disclosure teaches that, in some embodiments, the vasoconstricting agent is selected from the group consisting of prostaglandin F2, thromboxane A2, and thromboxane B2.

Furthermore, in some embodiments of the present disclosure, administering the stromal vascular fraction cell population to a subject comprises distributing the stromal vascular fraction cell population in the bone marrow of the subject. In still further embodiments of the present disclosure, distributing the stromal vascular fraction cell population in the bone marrow of the subject increases an amount of red blood cells, white blood cells, megakaryocytes, platelets or a combination thereof in the subject.

Additionally, the present disclosure is directed, in some embodiments, to a composition, comprising a population of stromal vascular fraction cells, the population of stromal vascular fraction cells including one or more macrophages. In certain embodiments, the composition is formulated for intravenous injection. And in some embodiments, the composition comprises a pharmaceutically-acceptable carrier.

The present disclosure also includes, in certain embodiments, a kit, comprising a container including a population of stromal vascular fraction cells. The kit may further comprise a syringe for injecting the population of stromal vascular fraction cells. In some embodiments, the population of stromal vascular fraction cells has been depleted of macrophages. And in other embodiments, the population of stromal vascular fraction cells has been enriched with macrophages isolated from a stromal vascular fraction of adipose tissue.

Further advantages of the presently-disclosed subject matter will become evident to those of ordinary skill in the art after a study of the description, Figures, and non-limiting Examples in this document.

### BRIEF DESCRIPTION OF THE FIGURES

FIG. 1 presents the percentage of nucleated cells (Syto blue-positive) fresh adipose stromal vascular fraction (SVF) isolates and CD11b⁺ cell-depleted adipose SVF cells (SVF-MΦΔ) by flow cytometry (gated dot plots are shown in FIG. 5). A significant proportion of macrophages (F4/80⁺ cells) are also positive for the M2 class marker CD301, which are significantly depleted in the SVF-MΦΔ fraction. Data are shown as the mean±standard error of the mean (s.e.m.) *P<0.05 determined by t test between the SVF and SVF-MΦΔ groups for n=3 isolation runs.

FIG. 2 shows a schematic of the experimental plan involving the treatment of the injured (cuffed) right saphenous artery of mice with syngeneic adipose SVF cells constitutively expressing luciferase and GFP reporter transgenes. Also shown are a gross view and a histological cross-section of a cuffed saphenous artery.

FIG. 3 provides hematoxylin and eosin (H&E) stained histological cross sections of normal (non-cuffed) and cuffed mouse saphenous arteries untreated or treated with adipose SVF cells or SVF cells depleted of CD11b⁺ cells (SVF-MΦΔ). Right-side panels are higher magnification images of the lower magnification images shown in the middle column panels.

FIG. 4 presents lumen diameters of untreated (n=9) and cell-treated cuffed saphenous arteries measured from histological sections. Cell treatments included complete SVF cell isolates (C+SVF, n=7), SVF isolates depleted of CD11b⁺-cells (C+SVF-MΦΔ, n=7), and the CD11b⁺-enriched cell fraction removed from the SVF isolate (C+MΦΔ, n=3). Data are shown as the mean±s.e.m. *P<0.05 determined by a one-way ANOVA.

FIG. 5 provides gated dot plots of select markers for the SVF cells and the corresponding CD11b⁺ cell-depleted fraction (SVF-MΦΔ) for one of three isolations. Cells were isolated from adipose collected from adult transgenic mice expressing GFP via the tie-2 gene promoter. The reported tie-2 expression is generated from the GFP channel and includes both low (circled population) and high expression levels. In all cases, plots were generated from nucleated (Syto blue-positive) cells to distinguish from possible debris or cell fragments generated during the digestion and isolation. The lower right panel of each set depicts the dot plot of CD301⁺ cells of only the gated set shown for F4/80⁺ cells.

FIG. 6 shows morphometric measurements of cuffed saphenous arteries in untreated (n=3) mice and mice treated with different SVF cell preparations made from histological cross sections. IEL and EEL represent the areas of the vessel circumscribed by the internal elastic lamina and external elastic lamina, respectively. Cell treatments included complete SVF cell isolates (C+ SVF, n=4), SVF isolates depleted of CD11b⁺-cells (C+SVF-Mac, n=3), and the CD11b⁺-enriched cell fraction removed from the SVF isolate (C+Mac, n=4). Data are shown as the mean±s.e.m. No significant differences were found by a one-way ANOVA.

FIG. 7 illustrates morphometric measurements of normal (un-cuffed) saphenous arteries in untreated (n=9) mice and mice treated with different SVF cell preparations made from histological cross sections. IEL and EEL represent the areas of the vessel circumscribed by the internal elastic lamina and external elastic lamina, respectively. Cell treatments included complete SVF cell isolates (C+SVF, n=7), SVF isolates depleted of CD11b⁺-cells (C+SVF-Mac, n=7), and the CD11b⁺-enriched cell fraction removed from the SVF isolate (C+Mac, n=3). Data are shown as the mean±s.e.m. No significant differences were found by a one-way ANOVA.

FIG. 8 provides vessel relaxation curves of isolated, normal saphenous arteries from mice untreated or treated with complete SVF cell isolates (SVF) or SVF isolates depleted of CD11b⁺-cells (SVF-MΦΔ). In all cases, measured internal vessel diameters were normalized to maximal diameters to account for inter-vessel variability. *P<0.05 determined by t test between the untreated and SVF-treated vessels at each pressure.

FIG. 9 provides a visualization of DCF epi-fluorescence in saphenous arteries mounted in the relaxation measurement rig to visualize the presence of H₂O₂. Arteries were from untreated, SVF-treated or SVF-MΦΔ-treated mice. The bottom row shows images of the same vessels in the top row after treatment with the tissue-permeant, stabilized PEGylated-catalase (Cat). All images were acquired at the same camera exposure and post-acquisition image processing settings.

FIG. 10 shows measurements of the intensity of DCF fluorescence for the different vessels shown in FIG. 9. P value was determined by one way ANOVA within each pre- and post-catalase group.

FIG. 11 presents normalized DHE fluorescence intensities of untreated and SVF-treated (n=3 for each) saphenous arteries mounted in the relaxation measurement rig to visualize the presence of O₂⁻. Further, in the right panel, FIG. 11 provides DAF fluorescence intensities of untreated and SVF-treated (n=3 for each) saphenous arteries mounted in the relaxation measurement rig to visualize the presence of NO.

FIG. 12 shows measurements of the density of DCF-bright cells within the vessel wall for the different vessels shown in FIG. 9. P value was determined by one way ANOVA within each pre- and post-catalase groups, n≧3 for each of the 6 experimental groups. All data are shown as the mean±s.e.m.

FIG. 13 presents vessel relaxation curves for isolated, normal saphenous arteries from mice treated with either L-NAME (to block nitric oxide production) or PEGylated-Catalase (to scavenge hydrogen peroxide). In all cases, measured internal vessel diameters were normalized to maximal diameters to account for inter-vessel variability.

FIG. 14 provides a visualization (enface view) of GFP⁺ cells (green) within regions of saphenous artery walls from untreated, SVF-treated or SVF-MΦΔ-treated mice. The lectin GS-1 (red) was used to identify the endothelium of the vas vasorum and infiltrated macrophages. The images shown are stills from volume rendered confocal stacks (rotating animation of rendered image stacks of an SVF-treated vessel). The lower left panel shows an end view of a normal, untreated artery to locate the vas vasorum relative to the internal (IEL) and external (EEL) elastic lamina (shown blue via hydrazide staining)

FIG. 15 illustrates are the density of GFP⁺ cells and the percent fraction of those cells also positive for GS-1 lectin in SVF-treated or SVF-MΦΔ-treated arteries (n=3 for all groups), as related to FIG. 14. Data are shown as the mean±s.e.m. P determined by t test.

FIG. 16 provides a visualization of SVF cells within histological paraffin sections of cuffed and normal saphenous arteries from untreated, SVF-treated and SVF-MΦΔ-treated mice via immunostaining for luciferase. The SVF sources were transgenic mice constitutively expressing both luciferase and GFP. Brown stain indicates positive luciferase immune-staining and the presence of SVF cells. Tissues were harvested 1 week after cell delivery.

FIG. 17 presents a visualization of SVF cells injected via the lateral tail vein into mice with cuffed saphenous arteries by bioluminescence. * indicates the limb in which the saphenous artery was cuffed. Shown are the cell distributions 1, 2, and 8 weeks following injection. Also shown are GFP⁺ cells (arrows) within the bone marrow of an SVF-treated, cuffed mouse 12 weeks after injection.

FIG. 18 is a visualization of luciferase-positive SVF cells within histological paraffin sections of different tissues from SVF-treated normal (non-cuffed) mice. Brown stain indicates positive luciferase immune-staining and the presence of SVF cells. Tissues were harvested 1 week after cell delivery. Positive and negative controls involved untreated transgenic luciferase-positive donor and wild type mice, respectively.

FIG. 19 provides a schematic depicting the proposed mechanism by which intravenously injected SVF cells lessen myogenic tone. Macrophages (red stars) within the injected and systemically circulating SVF cell isolate infiltrate the adventitia (A), but not the media (M) or intima (I) layers, via the vaso vasorum of insulted vessels and relax media smooth muscle by generating H₂O₂.

FIG. 20 shows the plasma concentration of IL-10 in blood collected from normal mice and mice with cuffed saphenous arteries untreated or treated with different SVF cell preparations. In normal (un-cuffed) mice, blood was collected 1 week after cell injection. In mice with cuffed vessels, blood was collected 2 weeks after cuffing, 1 week after cell injection. Data are shown as the mean±s.e.m. No significant differences were found by a one-way ANOVA.

FIG. 21 presents a visualization of one half of a region of a normal saphenous artery harvested from a mouse that had received an injection of syngeneic, GFP⁺ SVF cells (green) and stained en bloc with GS-1-rhodamine (red, to visualize the vaso vasorum) and hydrazide (blue, to visualize elastic matrix). Shown is a volume rendered confocal image stack of all three channels rotating around a vertical axis with the lumen-side facing up and showing the location of the vaso vasorum within the adventitia. The average distance between the internal and external layers of hydrazide staining (i.e. medial thickness) is ˜25 μm. The depicted vessel was harvested 1 week after intravenous injection of the SVF cells.

FIG. 22 provides histology and morphometry of saphenous arteries from untreated and cell-treated mice.

FIG. 23 presents cytometry histograms of anti-CD14 fluorescence in the total isolate (SVF) and the depleted isolate (SVF-MΦΔ) used to treat mice.

FIG. 24 illustrates lumen areas of saphenous arteries from vessel-cuffed and normal mice untreated or treated with different cell fractions (SVF-Mac=isolates depleted of MΦs, +Mac=MΦs only).

FIG. 25 shows diameter changes of isolated saphenous artery segments from un-treated and cell-treated (SVF) mice in response to acetylcholine, nitroprusside, and intraluminal pressures under conditions with (active) and without (passive) extra-vascular Ca⁺². Values between the two active groups in the myogenic experiments are different (p<0.05) at the 30 mmHg and higher pressures.

FIG. 26 shows peroxide levels (DCF fluorescence) in an untreated and a cell-treated saphenous artery (plus and minus catalase (Cat) to scavenge peroxide).

FIG. 27 illustrates the scavenging of H₂O₂ with catalase in cell (SVF)-treated vessels inhibited the cell-dependent change in myogenic tone.

FIG. 28 presents image slices from a confocal image stack showing GFP⁺ injected cells (arrows) within the adventitia a normal saphenous artery intravenous treated with total (SVF) and MΦ-depleted total (SVF-Mac) isolates.

### DETAILED DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional components or limitations described herein or otherwise useful.

The presently-disclosed subject matter includes compositions and methods for the treatment of blood vessel disease. In particular, the presently-disclosed subject matter relates to compositions and methods for the treatment of blood vessel disease that make use of a population of stromal vascular fraction cells. In some embodiments of the presently-disclosed subject matter, a composition is provided that comprises a population of stromal vascular fraction cells.

As used herein the phrase “population of stromal vascular fraction cells,” and grammatical variations thereof, is used to refer to a group of cells isolated from a stromal vascular fraction obtained from adipose tissue. Such stromal vascular fractions are known to those skilled in the art and are typically obtained by enzymatically digesting an amount of adipose tissue obtained from a subject, followed by a period of centrifugation to pellet the stromal vascular fraction of the adipose tissue. In this regard, the stromal vascular fraction contains a number of cell types, including pre-adipocytes, mesenchymal stem cells (MSCs), endothelial progenitor cells, T cells, B cells, mast cells, and adipose tissue macrophages, as well as small blood vessels or microvascular fragments found within the stromal vascular fraction.

It has now been determined, however, that with respect to the treatment of blood vessel disease, macrophages comprise an important therapeutic component of the stromal vascular fraction. As such, in some embodiments, a composition is provided that comprises a population of stromal vascular fraction cells including one or more macrophages. In certain embodiments, the present disclosure provides a composition comprising macrophages derived and/or isolated from the stromal vascular fraction of adipose tissue and methods of administering such compositions to a subject. In some embodiments, the present disclosure provides a composition comprising a population of stromal vascular fraction cells, wherein the population of stromal vascular fraction cells comprises an enriched fraction of macrophages derived from the stromal vascular fraction of adipose tissue. Indeed, the compositions of the present disclosure may comprise, in certain embodiments, an effective amount of macrophages derived from the stromal vascular fraction of adipose tissue.

The present disclosure further provides, in some embodiments, a pharmaceutical composition, comprising one or more macrophages isolated from the stromal vascular fraction of adipose tissue. In some embodiments, the composition comprising said macrophages is administered to a subject intravenously.

For further explanation and guidance regarding the disassociation of adipose tissue to produce a stromal vascular fraction, see, e.g., U.S. Pat. No. 4,820,626, the entire contents of which are incorporated herein by this reference.

With respect to the formulations of the compositions of the presently-disclosed subject matter, in some embodiments, the compositions described herein are formulated as pharmaceutical compositions that comprise the compositions and a pharmaceutically-acceptable vehicle, carrier or excipient.

In some embodiments, the compositions are formulated for intravenous injection, and can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compounds can also be administered by the drip method, whereby a formulation including a pharmaceutical composition of the present invention and a physiologically-acceptable excipient is infused. Physiologically-acceptable excipients can include, for example, 5% dextrose, 0.9% saline, Ringer's solution or other suitable excipients.

Further provided, in some embodiments of the presently-disclosed subject matter are methods for treating a blood vessel disease. In some embodiments, a method for treating a blood vessel disease is provided that comprises administering to a subject an effective amount of a composition comprising a stromal vascular fraction cell population. In some embodiments, the stromal vascular fraction that is administered to the subject is isolated directly from adipose tissue of the subject prior to its administration back to the subject to treat the blood vessel disease.

In some embodiments, to isolate the stromal vascular fraction from the adipose tissue and remove cell clumps found in the adipose tissue, a sequential screening methodology is used whereby the stromal vascular fraction isolate is sequentially screened a number of times through filters. In some embodiments, the fraction is screened through a filter having a pore size of between about 10 and about 100 microns. In some embodiments, the fraction is screened through a filter having a pore size of about 20 to about 90 microns. In some embodiments, the fraction is screened through a filter having a pore size of less than 50 microns. In certain embodiments, the fraction is screened through a filter having a pore size of about 40 microns. In further embodiments, the fraction is screened sequentially through multiple filters, and in certain embodiments, the last of the sequential filters has a pore size of about 40 microns or less.

As used herein, the terms “treatment” or “treating” relate to any treatment of a cardiovascular disease, including but not limited to prophylactic treatment and therapeutic treatment. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. As such, the terms “treatment” or “treating” include, but are not limited to: preventing a blood vessel disease or the development of a blood vessel disease; inhibiting the progression of a blood vessel disease; arresting or preventing the further development of a blood vessel disease; reducing the severity of a blood vessel disease; ameliorating or relieving symptoms associated with a blood vessel disease; and causing a regression of a blood vessel disease or one or more of the symptoms associated with a blood vessel disease.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the present invention include, but are not limited to, systemic administration, parenteral administration (including intravascular, intramuscular, intraarterial administration), oral delivery, buccal delivery, rectal delivery, subcutaneous administration, intraperitoneal administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

In some embodiments of the presently-disclosed subject matter, administering the therapeutic composition to the subject comprises intravenously injecting the composition comprising the stromal vascular fraction cell population into a subject. In some embodiments, administering the stromal vascular fraction cell population to the subject comprises distributing the stromal vascular fraction cell population in the intima, media, or adventitia of a blood vessel of the subject or in a combination thereof. In some embodiments, the stromal vascular fraction cell population is distributed to the bone marrow of a subject. In some embodiments, the stromal vascular fraction cell population is distributed to the intima, media, and/or adventitia of a blood vessel and/or to the bone marrow as a result of intravenously injecting the stromal vascular fraction cell population into the subject. In some embodiments, instead of homing to the site of inflammation, the stromal vascular cell populations repopulate normal or non-inflamed blood vessels.

The present disclosure further provides, in some embodiments, a method for treating, preventing or reducing the formation of an intimal lesion, wherein the method comprises administering a composition comprising the stromal vascular fraction cell population to a subject. In some embodiments, the present disclosure provides a method for inducing vasodilation by administering to a subject an effective amount of a composition comprising the population of stromal vascular fraction cells. In certain embodiments, the present disclosure further provides a method for reducing local and/or systemic inflammation in a subject, wherein the method comprises administering to the subject a composition comprising a population of stromal vascular fraction cells. And in still further embodiments, the present disclosure provides a method for treating microvascular disease in a subject, wherein the method comprises at least the step of administering to the subject a composition comprising a population of stromal vascular fraction cells. Additional embodiments provide a method for improving distal vessel function in a subject by administering to the subject a composition comprising a population of stromal vascular fraction cells. The present disclosure also provides a method for modulating vascular myogenic activity, comprising the step of administering, to a subject, a composition comprising a population of stromal vascular fraction cells. In some embodiments of the present disclosure, the population of stromal vascular fraction cells is administered to a subject intravenously. And in certain embodiments, the population of stromal vascular fraction cells comprises at least one macrophage. Further, the population of stromal vascular fraction cells comprises, in some embodiments, at least one adipose-derived macrophage.

Regardless of the route of administration, the compositions of the presently-disclosed subject matter are typically administered in amount effective to achieve the desired response. As such, the term “effective amount” is used herein to refer to an amount of the therapeutic composition (e.g., a composition comprising a stromal vascular fraction cell population and a pharmaceutically acceptable vehicle, carrier, or excipient) sufficient to produce a measurable biological response (e.g., an increase in an amount of vasodilation). For example, a “therapeutically effective amount” refers to an amount that is sufficient to achieve the desired therapeutic result, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compositions employed; the duration of the treatment; drugs used in combination or coincidental with the specific compositions employed and like factors well known in the medical arts. For example, it is well within the skill of the art to start doses of a composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Actual dosage levels of active ingredients in a therapeutic composition of the present disclosure can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

In some embodiments of the therapeutic methods described herein, administering the stromal vascular fraction cell population to the subject increases an amount of a vasodilatory agent in the subject, which, in turn, can increase an amount of vasodilation in a subject. In some embodiments, administering the composition increases the activity of a vasodilatory agent in the intima media and adventitia of a blood vessel. In some embodiments, the vasodilatory agent is selected from the group consisting of nitric oxide, histamine, prostacyclin, prostaglandin E2, prostaglandin I2, leukotriene C4, leukotriene D4, leukotriene E4, vasoactive intestinal peptide (VIP), adenosine, adenosine triphosphate, adenosine diphosphate, L-arginine, bradykinin, substance P, nicotinic acid, platelet activating factor, carbon dioxide, lactic acid, natriuretic peptide, heparin, heparin sulfate, and endothelium-derived hyperpolarizing factor.

In further embodiments of the therapeutic methods, administering the composition to the subject decreases an amount of vasoconstriction in a blood vessel of the subject. In some embodiments, administering the composition decreases the activity of a vasoconstricting agent in a blood vessel of a subject. In some embodiments, the vasoconstricting agent is chosen from adrenaline, epinephrine, prostaglandin F2, thromboxane A2, and/or thromboxane B2.

In further embodiments, wherein administering the stromal vascular fraction cell population to the subject comprises distributing the stromal vascular fraction cell population in the bone marrow of the subject, distributing the stromal vascular fraction cell population in the bone marrow of the subject increase an amount of red blood cells, white blood cells, megakaryocytes, platelets or a combination thereof in the subject. In certain embodiments, the stromal vascular fraction cell population is administered systemically to a subject.

In some embodiments, small artery function is improved by administration of the stromal vascular fraction cell population. In certain embodiments, administration of the stromal vascular fraction cell population reduces myogenic tone. Furthermore, because the stromal vascular fraction cell populations are normal resident tissue cells, this pro-vascular behavior likely reflects an intrinsic tissue homeostasis activity that is enriched by the administration of the stromal vascular fraction cell population.

The present disclosure provides an adipose-derived, systemic, cell-based therapy that potentiates small vessel vasodilation in mice. In certain embodiments, this therapy involves a mixed population of homeostatic stromal and vascular cells comprised of endothelial cells, perivascular cells, mesenchymal cells, resident macrophages/monocytes, and other immune cells. In some embodiments, CD11b⁺ cells within this isolate provide a positive vasoactive effect that involves a reduction in myogenic tone associated with increased levels of H₂O₂. Accordingly, in certain embodiments, the stromal vascular fraction cell population comprises CD11b-positive (CD11b⁺) cells; and in some embodiments, the CD11b-positive cells are administered to a subject systemically. Indeed, the present disclosure provides that the CD11b-positive cells within the adipose stromal vascular fraction improve small artery function by reducing myogenic tone when delivered systemically.

Moreover, in some embodiments, the cells delivered via intravenous injection persistently populate the adventitia of peripheral arteries and other tissues. Thus, in certain embodiments, this ready source of therapeutic cells is used to treat distal vessel dysfunction, particularly in diffuse disease.

In some embodiments, the present disclosure provides that intravenously-delivered, freshly-isolated, adipose stromal vascular fraction (SVF) cells can widely disseminate into a variety of tissues, including the adventitia of small arteries, and potentiate vasodilation of the saphenous artery injured by a focal inflammatory insult. In certain embodiments, this fresh isolate is a mixed population of homeostatic cells, comprising relatively few mesenchymal stem cells. In some embodiments, the CD11b⁺ cells within this isolate, of which a majority may be positive for M2 macrophage markers, enhance the vasoactive effect, which involves a reduction in myogenic tone associated with increased levels of hydrogen peroxide. Thus, this ready source of cells may be used to treat distal vessel dysfunction, particularly in diffuse disease.

Still further provided, in some embodiments of the presently-disclosed subject matter are kits including a therapeutic composition described herein. In some embodiments, a kit is provided that comprises a first container including a population of stromal vascular fraction cells, the population of stromal vascular fraction cells being depleted of macrophages. In other embodiments, a kit is provided that comprises a first container including a population of stromal vascular fraction cells, wherein the population of stromal vascular fraction cells has been enriched with additional macrophages derived from a stromal vascular fraction of adipose tissue. In some embodiments, a second container is provided that includes a vehicle for use in injecting the compositions. In some embodiments, the kits of the presently-disclosed subject matter further comprise instructions and/or a syringe for injecting the population of stromal vascular fraction cells into the subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals that are kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

Additionally, the terms “subject” or “subject in need thereof” refer to a target of administration, which optionally displays symptoms related to a particular disease, pathological condition, disorder, or the like. The terms do not denote a particular age or sex. Thus, adult and newborn subjects, as well as fetuses, whether male or female, are intended to be covered. A “patient” refers to a subject afflicted with a disease or disorder.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Polynucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

### EXAMPLES

The presently-disclosed subject matter is further illustrated by the following specific but non-limiting examples. The examples may include compilations of data that are representative of data gathered at various times during the course of development and experimentation related to the presently-disclosed subject matter.

### Example 1

The relatively few therapeutic options for treating small vessel disease create a significant clinical challenge. The stromal vascular fraction (SVF) derived from adipose tissue is a rich source of regenerative cells shown to have anti-inflammatory and vascular reparative capabilities. The ability of these SVF cells to reverse vascular insult was thus explored in a murine model of artery inflammation induced by the placement of a 2 mm long polyethylene cuff around the left femoral artery in male wild type FVB/n mice. One week after cuffing, freshly isolated SVF cells harvested from syngeneic FVB/n^{Tg(CAG-luc,-GFP)} mice ubiquitously and constitutively expressing the firefly luciferase and green fluorescent protein genes were injected via a tail vein. Sham mice, which underwent femoral exposure without receiving a cuff, were treated identically. Bioluminescence confirmed delivery and the persistent presence of SVF cells. Histological analysis of vessels indicated cuffed femoral arteries were surrounded by granulation tissue and exhibited intimal lesions associated with significant medial inward remodeling. In contrast, intravenous delivery of SVF cells after lesion formation, regardless of the dose, lessened intimal lesions, reversed medial remodeling, and reduced perivascular fibrosis. Immunostaining for luciferase identified SVF cells throughout the vessel wall and the surrounding extra-vascular tissue that were also positive for markers of M1 and M2 macrophages. In conclusion, intravenous delivery of freshly isolated adipose SVF cells reversed the vascular insult and peri-vascular granulation tissue due to inflammation.

Based on these results, and without wishing to be bound by any particular theory, it is believed that a therapeutic approach based on macrophage-rich adipose stromal vascular fraction cells can be used to treat any disease in which distal blood flow (below the large supply arteries) is compromised. While some examples provided herein are from the perspective of peripheral artery disease in which large arteries of the upper leg are repaired, yet poor blood flow to the lower limb and foot persists, there is broad applicability. Additionally, it is believed that the therapeutic aspects depend, at least in part, on a macrophage presence as well as intravenous delivery. In addition to targeting vascular health, the SVF cells also populate bone marrow (and likely other tissue beds), providing a means to reconstitute bone marrow or supplement bone marrow regeneration. In this regard, possible relevant disease conditions that can be treated by the foregoing methodology and compositions include, but are not limited to: ischemia; Reynaud's disease; Buerger's disease; hypertension; chemotherapeutic compromise (vessel dysfunction secondary to chemo treatments or immuno suppressants); erectile dysfunction; inflammation; atherosclerosis; and infection.

Using a mouse model of small artery insult, a cell-based therapy has been identified that potentiates small vessel function by facilitating vasodilation. Unlike more conventional therapeutic cell preparations, in certain embodiments, this cell therapy involves a mixed population of adipose stromal and vascular cells comprised of endothelial cells, pericytes, mesenchymal stem cells, resident macrophages/monocytes, and other immune cells. Preliminary observations indicate that selective depletion of macrophages from the total cell preparation abolishes the pro-vascular effect. Furthermore, vasoactive arteries isolated from cell-treated mice respond normally to increasing doses of select vasodilators but exhibit reduced myogenic tone as compared to untreated arteries. Moreover, cells of the therapeutic preparation populate the media and adventitia of peripheral arteries in treated mice.

### Materials and Methods

All animal studies were performed under protocols approved by the University of Louisville Institutional Animal Care and Use Committee (IACUC) and according to the National Institutes of Health Guidelines for the Care and Use of Laboratory Animals. Mouse strains used in the study include FVB/n, FVB-Tg(CAG-luc,-GFP)L2G85Chco/J (Cao, et al., 2004), and Tg(TIE2GFP)287Sato/J (Motoike, et al., 2000), either purchased from The Jackson Laboratory (Jax Labs) or obtained from in-house colonies. All mice (donors and recipients) were males between 10 and 23 weeks of age.

Saphenous Artery Cuffing: All procedures were performed using sterile technique and reflect modifications to a previously published method (Moroi, 1998). The medial area of the right hind limb of an anesthetized, supine mouse maintained at 37° C. body temperature was depilated and wiped with Novalsan™ solution. A 1 cm incision on the calf-side of the midline approximately ⅓ the distance proximal to the knee was made and deflected to the midline to expose the femoral-saphenous vessels. The overlying fascia was blunt-dissected to expose the vascular-nerve sheath, which was further opened to access the vessels. The saphenous artery was carefully freed from the saphenous vein for a length of 4 mm. A 2 mm long section of ETO-sterilized polyethylene-50 (PE-50) tubing split length-wise down the middle was placed around the freed artery section and tied closed with 5-0 silk ligature. The skin was placed back into position and closed with a single surgical clip. Sham groups were prepared as described above except the cuff was not placed.

Isolated of Adipose SVF Cells: For SVF isolations, uterine horn or epididymal fat pads from female or male mice, respectively, were collected, weighed, minced until homogenous and paste-like, and digested with a filter sterilized solution of 2 mg collagenase and 1 mg DNAse per mg fat in a 1:1 volume of 0.1% BSA-DCF-PBS to fat collected. The resulting final concentration is 0.1% (w/v) collagenase +0.05% (w/v) DNAse in 0.1% BSA-DCF-PBS in 50% (v/v) collagenase solution/tissue homogenate. The resulting fat-collagenase mixtures were placed into separate 50 ml conical centrifuge tubes with Teflon® coated magnetic stir bars and agitated (Envirogenie®, Scientific Industries Inc.) at 35 rpm rotation for 35 minutes at 37° C. After digestion, the digestate was centrifuged at 400×g for 4 minutes to pellet the SVF cells from the adipocytes, which was aspirated and discarded. The SVF pellets were suspended and washed in 0.1% BSA-DCF-PBS twice, each time collecting the pellet by centrifugation at 200×g for 3 minutes. The washed pellet, suspended in 10 ml of 0.1% BSA-DCF-PBS, was passed through a 20 μm nylon screen pre-wetted with 0.1% BSA-DCF-PBS to remove incompletely digested matrix fragments and clumped cells. The flow through of single cells was collected and counted with a NucleoCounter®. Total SVF cell yields averaged 2.58×10⁶±0.46×10⁶ cells/gm of fat.

Magnetic Depletion of SVF Cell Isolates: Depletion experiments utilized the Miltenyi MACS system according to the manufacturer's instructions. Briefly, to deplete SVF cell isolates of CD11b⁺ cells, up to 10⁷ screened, SVF cells were suspended in 90 μl MACS buffer (degassed solution of PBS pH 7.2, 0.5% BSA, and 2 mM EDTA stored at 4° C.) plus 10 μl of anti-mouse CD11b antibody conjugated to iron particles (Miltenyi, cat #130-049-601) and incubated at 4° C. for 15 minutes. Afterwards, an additional 3 ml MACS buffer was added to the cell suspension and centrifuged at 400×g for 4 minutes to pellet again. The supernatant was aspirated and the cell pellet re-suspended with 1.0 ml MACS buffer, while the magnet and Biotec MACS column stored at −20° C. were setup and pre-wetted with 0.5 ml MACS buffer before loading the SVF supernatant 0.5 ml at a time. The cell-loaded column was allowed to drain completely then flushed with 0.5 ml MACS buffer at least three times to remove additional cells. The effluent was collected and considered to be the CD11b⁺-depleted fraction (SVF-MΦΔ) which represented a 38.1%±1.65 reduction in total cell numbers. Cells in all fractions were counted with a NucleoCounter®.

Tail Vein Injection of Cells: SVF cell preparations were suspended in 0.2 ml of sterile, injectable saline per mouse for tail vein injection and loaded into a tuberculin syringe. Using a 30 g needle, the entire cell suspension was injected into the venous blood supply via the lateral tail vein, which was pre-warmed with a heat lamp at the following dosages: total SVF cells at 1×10⁶ cells per mouse; SVF-MΦΔ cells at 8×10⁵ cells per mouse; and MIO cells at 2×10⁵ cells per mouse.

Histologic Evaluation and Immunostaining: At explant, anesthetized mice were cannulated with a PE 50 catheter through the left ventricle and perfused with 10 ml of warmed PBS containing a vasodilator cocktail (2.5 μg/ml SNP, 60 μg/ml papaverine, 10 U/ml heparin, and 1 mg/ml adenosine) and then with 12 ml of warm 4% paraformaldehyde/PBS all at 100 mmHg pressure. A small opening was made in the left atrium as a circulatory exit for the perfusate. Harvested tissue samples were placed in 4% paraformaldehyde/PBS and kept at 4° C. until processed into paraffin prior to sectioning and staining For morphometry, sections were stained with hematoxylin and eosin using standard methods. To identify injected cells in saphenous arteries and select tissues, sections were immunostained for luciferase-positive cells using an HRP kit (Dako EnVision+ System HRP) according to manufacturer's instructions. Sections were treated with 3% hydrogen peroxide for 10 min at room temperature prior to incubation with an anti-firefly luciferase antibody (Abcam) at a 1:1000 dilution in 10% goat serum (Sigma) for an hour at room temperature. All sections were then counterstained with hematoxylin and coverslipped. Tissue sections from luciferase-positive donor mice and wild type mice that did not receive cells served as positive and negative controls, respectively, for immunostaining.

Flow Cytometry: Aliquots of SVF cells and CD11b⁺-depleted SVF cells (SVF-MΦΔ) isolated from tie2:GFP expressing transgenic mice were divided into polypropylene tubes for flow cytometry at a concentration of 5×10⁵-1×10⁶ cells in 100 μl Wash Buffer (DPBS containing 1% BSA and 0.025M HEPES) per tube. Aliquots of the following antibodies (to yield the optimized antibody dilution) were added to label cell surface markers: CD2-PE (BD 553112), CD45-PerCP (BD 557235), CD11b-APC (Miltenyi Biotec 130-098-088), Ly6G (Gr-1)-PE (Ebioscience 12-5931-83), FεR1-PerCP (Ebioscience 46-5898-82), CD11b-PE (Miltenyi Biotec 130-098-087), CD80-APC (Ebioscience 17-0801-82), F4/80-PerCP-Cy5.5 (eBioscience 45-4801), and CD301-AlexaFluor 647 (AbD Serotec MCA2392A647T). The GFP fluorescence was used to mark endothelial cells. Species-matched isotypes were added to separate tubes of FVB/n SVF cell isolates. Additionally, single color tubes of FVB/n SVF were used as compensation controls. Cells were incubated in antibodies at 4° C. for 30 minutes protected from light, lysed with BD PharmLyse (BD 555899) for 3 minutes at 37° C., washed twice with 2 ml Wash Buffer, spun at 350×g for 5 minutes to pellet, then suspended in 400 μl Wash Buffer per tube and analyzed on a BD LSRII flow cytometer using BD FACS Diva software. Post-acquisition data analyses were performed using FlowJo 7.6.2 software.

Saphenous Artery Vasoactive Responses: Saphenous arteries were explanted from anesthetized (5% isoflurane/O₂ balance) non-cuffed FVB/n mice with or without different cell treatments (Normal, SVF-injected, and SVF-MΦΔ-injected). The saphenous artery was placed in cold, filtered physiological saline solution (PSS, pH 7.4 containing 145 mM NaCl, 4.7 mM KCl, 2.0 mM CaCl₂, 1.17 mM MgSO₄, 1.2 mM NaH₂PO₄, 5.0 mM glucose, 2.0 mM pyruvate, 0.02 mM EDTA, 3.0 mM MOPS buffer, and 1% BSA). Arteries were cannulated on size- and resistance-matched glass pipettes in a Lucite chamber containing warm (37° C.) PSS as previously described (Kang, et al., 2011; LeBlanc, et al., 2010). Once arteries were cannulated and determined to be free of leaks, the chamber was placed on an Olympus IX51 inverted microscope with a side-mounted camera and calibrated electronic caliper measurement system (Colorado Video, Boulder, Colo.) attached. Intraluminal pressure was maintained at 50 mmHg using a servo controlled peristaltic pump (Living Systems Instrumentation, Burlington, Vt.), and vessels were visualized using a 10× objective for the rest of the experiment, unless otherwise noted. Arteries were pre-constricted with phenylephrine (2 μM) to approximately 30% of resting diameter. Vessels that did not constrict were discarded. To assess active myogenic response, the intraluminal pressure was decreased to 1 mmHg and sequentially raised (waiting 3 minutes at each step) while simultaneously recording luminal diameters throughout the procedure. Following active myogenic responses, intraluminal pressure was returned to 50 mmHg, the chamber was washed with fresh PSS, lumen diameter was measured pre- and post-addition of phenylephrine as previously described, and upon tone establishment, lumen diameter changes were recorded during drug dose response curves for Acetylcholine (doses ranged from 1×10⁻⁹ to 1×10⁻⁴ M, 3 minutes per dose) and, following a wash, for Sodium Nitroprusside (doses ranged from 1×10⁻¹⁰ to 1×10⁻⁴ M, 3 minutes per dose). The chamber was then washed with PSS without CaCl₂ to allow the vessels to maximally dilate; lumen diameter was recorded for each of four 15 minute washes, with vessel wall diameter measured during the second wash. Following the fourth wash without calcium, passive myogenic responses were measured for the same pressures previously described for the active myogenic response. Intraluminal diameters (measured with electronic calipers) were normalized to the maximum diameter obtained in the absence of calcium and reported as % relaxation: (diameter/max. diameter)×100.

PEG-Catalase Myogenic Responses and ROS Fluorescence Imaging: A separate group of saphenous arteries from SVF-injected and untreated FVB/n mice were used for ROS experiments. Following the active myogenic response assessment (as described above), the chamber was washed with PSS without albumin and vessels were incubated 10 minutes in the dark intra- and extraluminally with 5-(and-6)-choromethyl-2′,7′-dichlorodihydrofluorescein diacetate (DCF, 5 μM) to fluorescently measure the presence of H₂O₂ in the vessel walls (Kang, et al., 2011; Phillips, et al., 2007). Following DCF incubation, vessels were washed intra- and extraluminally with PSS without albumin. DCF fluorescence was visualized using an Olympus FITC filter (excitation=475 nm, emission=515 nm) with a 10× objective (FITC exposure time=5 ms) and a 4× objective (FITC exposure time=10 ms). Vessels were then incubated for one hour in PSS+albumin+PEG-Catalase (PEG-CAT, Sigma C4963, 500 U/ml) (Phillips, et al., 2007). Following PEG-CAT treatment, the chamber was washed with PSS without albumin and DCF was added intra- and extraluminally as before. DCF fluorescence images were captured with the same exposure times and magnifications, vessels were washed with calcium-free PSS+PEG-CAT for one hour as previously described, and passive myogenic responses were recorded. 10× and 4× pre- and post-PEG-CAT images were analyzed for FITC intensity within a 10×100 μm region of interest in both the left and right vessel wall using Nikon Elements software (Nikon Instruments, Melville, N.Y.).

A separate group of FVB/n normal mice were used to measure the levels of two additional ROS, NO and O₂⁻. Sections of both saphenous arteries were explanted from these animals. The left saphenous was cannulated and pressurized as previously described in PSS without albumin. The vessel was infused intraluminally with dihyodroethidium (DHE, 10⁻⁴ M) for 10 minutes. Both the vessel lumen and the chamber were washed with PSS without albumin. DHE is known to permeate cell membranes; when oxidized by O₂⁻, DHE is converted to fluorescent ethidium bromide that intercalates into nuclear DNA⁵. Following the PSS without albumin wash, 10× images of ethidium bromide fluorescence (excitation=540 nm, emission=605 nm, exposure time=40 ms) and hydroethidine fluorescence (excitation=350 nm, emission=460 nm, exposure time=200 ms) were captured and subsequently analyzed in Nikon Elements software by subtracting the hydroethidine fluorescence signal from the ethidium bromide fluorescence signal. The resulting ethidium bromide only image allowed for intensity measurements of O₂⁻ levels within a 40×150 μm region of both the left and right vessel wall. The right saphenous arteries from the same animals were subsequently cannulated and pressurized, constricted with PE, and myogenic responses were measured as previously described. Following myogenic responses, vessels were infused with 4-amino-5-methylamino-2′2′-difluorofluorescein diacetate (DAF, 5 μM) for 10 minutes protected from light, then lumen and chamber were washed with PSS without albumin. DAF fluorescence was visualized using an Olympus FITC filter (excitation=475 nm, emission=515 nm) with a 10× objective (FITC exposure time=30 ms) and a 4× objective (FITC exposure time=50 ms). After imaging, vessel was treated with N^{G}-nitro-L-arginine methyl ester (L-NAME; 10 μM) for 30 minutes and DAF fluorescence imaging was repeated⁴. After post-L-NAME imaging, chamber was washed with PSS+albumin, vessel was constricted with PE and post-L-NAME myogenic responses were measured. Passive myogenic responses in calcium-free solution were performed as previously described. Pre- and post-L-NAME DAF images were analyzed for fluorescence intensity in a 40×150 μm region of interest in both the left and right vessel wall using Nikon Elements software.

Confocal Microscopy: Fixed saphenous arteries (2% paraformaldehyde) from control FVB/n, SVF-, SVF-MΦΔ, and MΦΔ-injected mice were washed for 30 minutes in PBS, then permeabilized in PBS+0.1% Triton-X (Sigma, #T8787) for one hour. Vessels were washed for 5 minutes in PBS, then incubated overnight at 4° C. in rhodamine-conjugated Griffonia simplicifolia lectin I (GSL I, Vector Laboratories, #RL-1102, 1:200), an endothelial cell marker. The next day, arteries were washed for 2.5 hours in PBS at room temperature, protected from light. Following the wash, vessels were stained with Alexa 633 hydrazide (Molecular Probes, #A-30634, 0.2 μM), to visualize the elastin matrix (Clifford, et al., 2011), for 20 minutes at room temperature and protected from light. Stained vessels were placed in Fluoromount-G (Southern Biotech, #0100-01) on a dimpled slide to allow the vessels to retain their natural shape during imaging. Confocal imaging was performed on an Olympus FV1000 confocal microscope equipped with 488 (for visualizing GFP+ SVF cells), 543 (GSL I), and 633 (hydrazide) laser lines. Confocal stacks were obtained (1-1.5 μm step size) at 40× magnification through half of the vessel. Images were then opened in Amira software (VSG, Burlington, Mass., USA) and volume rendered.

Bioluminescence Imaging: In vivo bioluminescence imaging was performed on mice using a Photon Imager (Biospace Lab, Paris, France). Each mouse was injected intraperitoneally with D-luciferin potassium salt dissolved in PBS at a dose of 150 mg per kg of body weight. Mice were anesthetized with 3% isoflurane at 1 L/min in the beginning; then 2% isoflurane at 500 cc per minute was used to keep mice anesthetized during imaging. Mice were kept anesthetized with isoflurane and warm at 37° C. for the entire imaging period. Mice were imaged 10 minutes after D-luciferin injection. Signal intensity was quantified as the sum of all detected photon counts within the region of interest after subtraction of background luminescence.

### Results

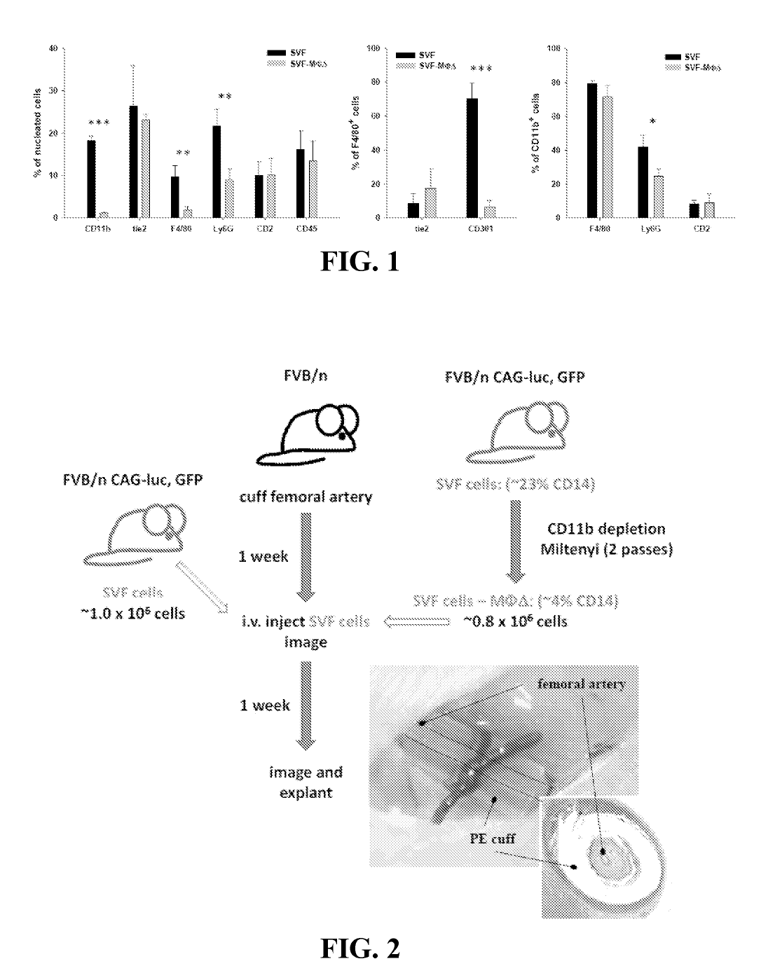

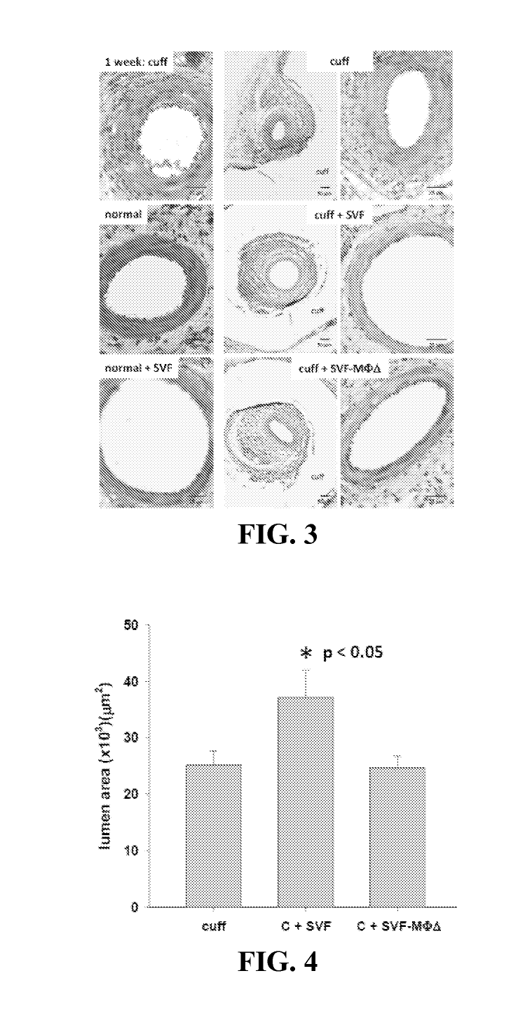

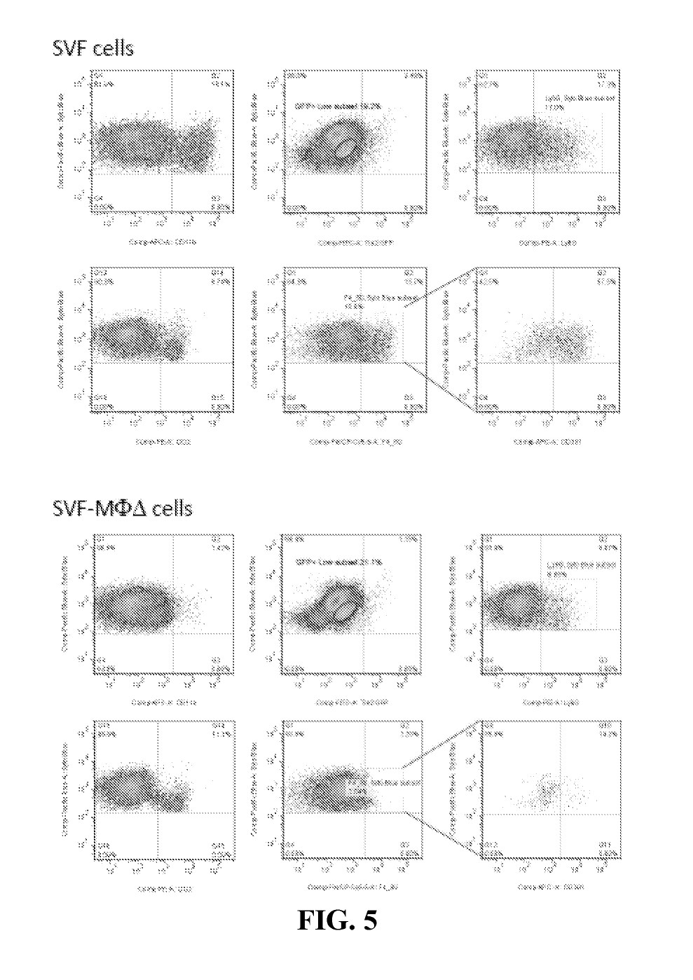

Non-vascular, homeostatic cells residing within the adventitia of arteries contribute to normalized vessel function (Majesky, et al., 2012; Dimayuga, et al., 2005). Freshly isolated, adipose stromal vascular fraction (SVF) cells (Gimble, et al., 2010) contain a heterogeneous mix of vascular, tissue, and resident-immune cells (FIG. 1, FIG. 5), many of which are similar to the homeostatic cells present within artery walls (Majesky, et al., 2012; Dimayuga, et al., 2005). Accordingly, it was suggested that intra-wall cellular dynamic could be enhanced to re-establish vessel function following vascular insult, we explored the ability of the SVF cells, as a potential source for such vascular homeostatic cells, to disseminate throughout the vasculature, take up residence within artery walls, and regulate vasoactivity. Further, a model of local vascular injury (Dimayuga, et al., 2005) was used in which the right medial saphenous artery of an adult FVB/n mouse was loosely cuffed with a short piece of PE tubing to induce a materials-driven inflammation leading to vascular insult (FIG. 2). After the vessel insult had developed (1 week with the cuff in place), 1×10⁶ freshly isolated SVF cells harvested from syngeneic donor reporter mice constitutively expressing luciferase and GFP were intravenously delivered (Cao, et al., 2004). One week later, mice were perfusion-fixed at 100 mm Hg pressure in the presence of a vasodilator cocktail, and the vessels were explanted. It was clear that the saphenous artery lumen diameter in the cuffed area was significantly increased following SVF cell treatment (FIG. 3, FIG. 4).

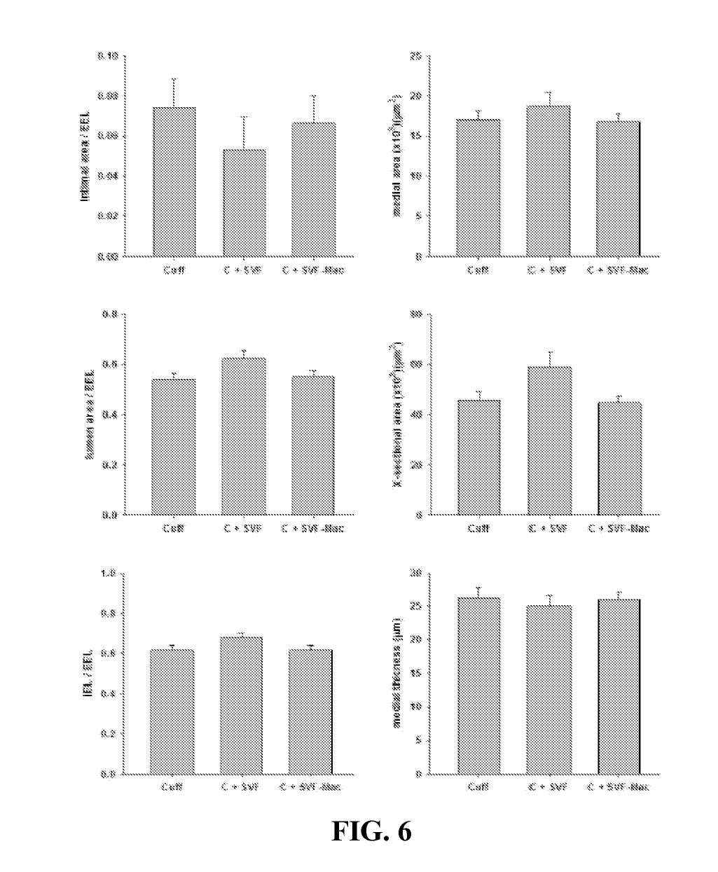

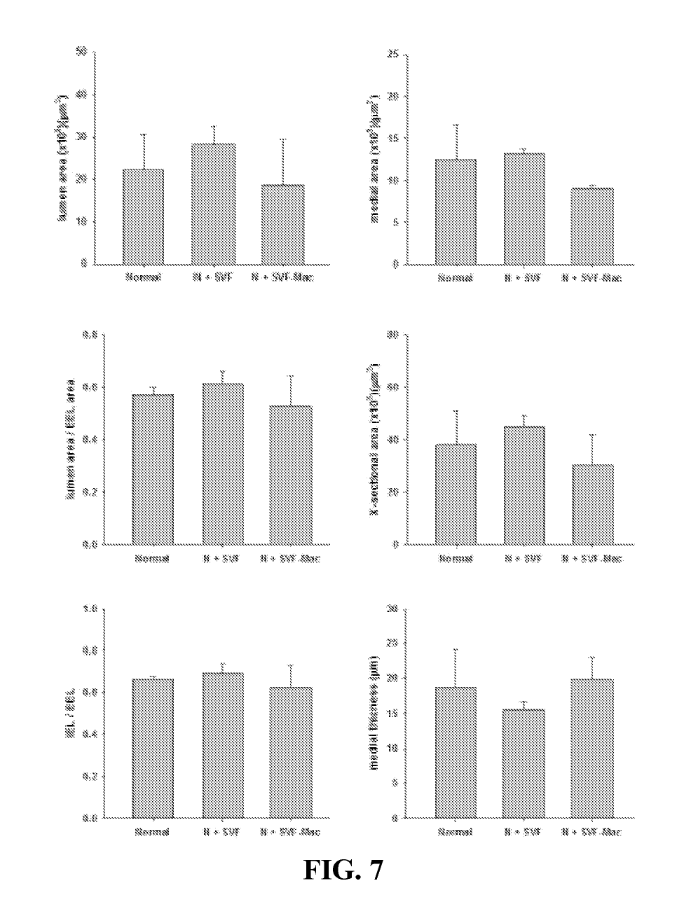

Normal, lean adipose is rich in macrophages (MΦs), most of which are pro-healing, homeostatic subtypes (Morris, et al., 2011; Murray, et al., 2011). Consequently, and because MΦs represent a relatively large fraction of the SVF isolate (FIG. 1), it was next determined if MΦs within the delivered SVF mediate the vasodilation. Depleting MΦs from the total isolate (FIG. 1) by selectively targeting the removal of CD11b⁺ cells (which is expressed predominately by monocytes/macrophages (Christensen, et al, 2001)) prior to intravenous injection into cuffed mice (FIG. 5) attenuated the artery dilation promoted by the SVF cell isolate (FIG. 3, 4). As with the SVF cell treatment, there were no significant changes in other artery morphometric parameters in these mice treated with the depleted SVF isolate (SVF-MΦΔ fraction) (FIG. 6). Delivery of just the CD11b⁺ cell population (MΦΔ fraction) tended to reverse this attenuation (FIG. 4). Similar findings were observed for saphenous arteries from normal (i.e. non-cuffed), cell-treated mice (FIG. 7).

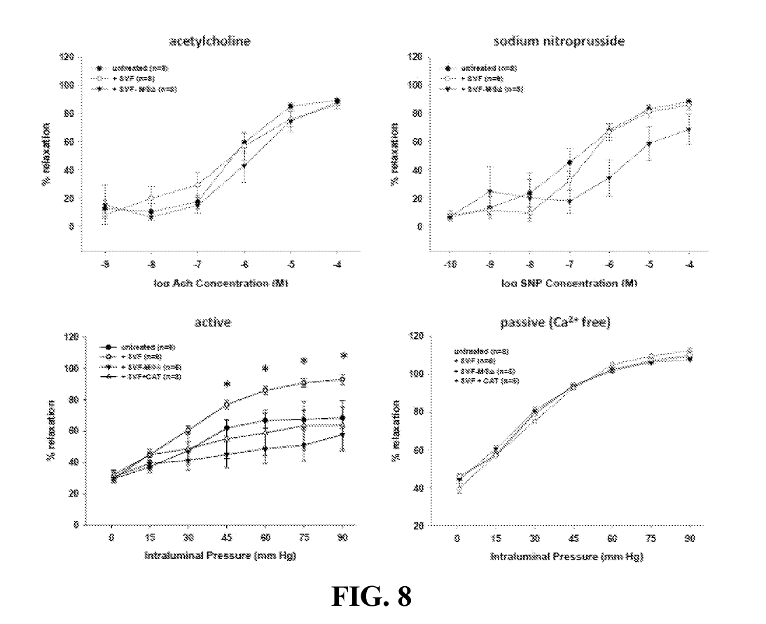

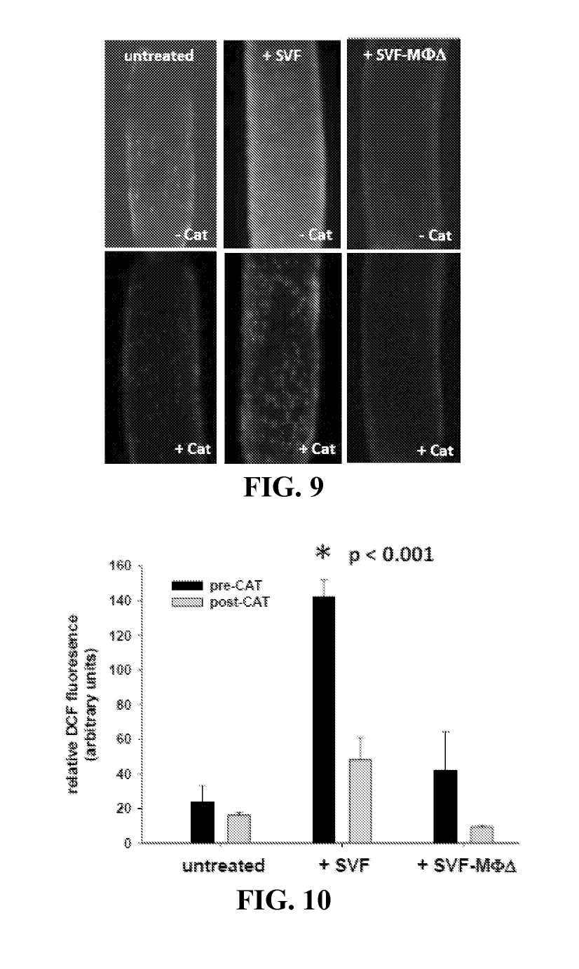

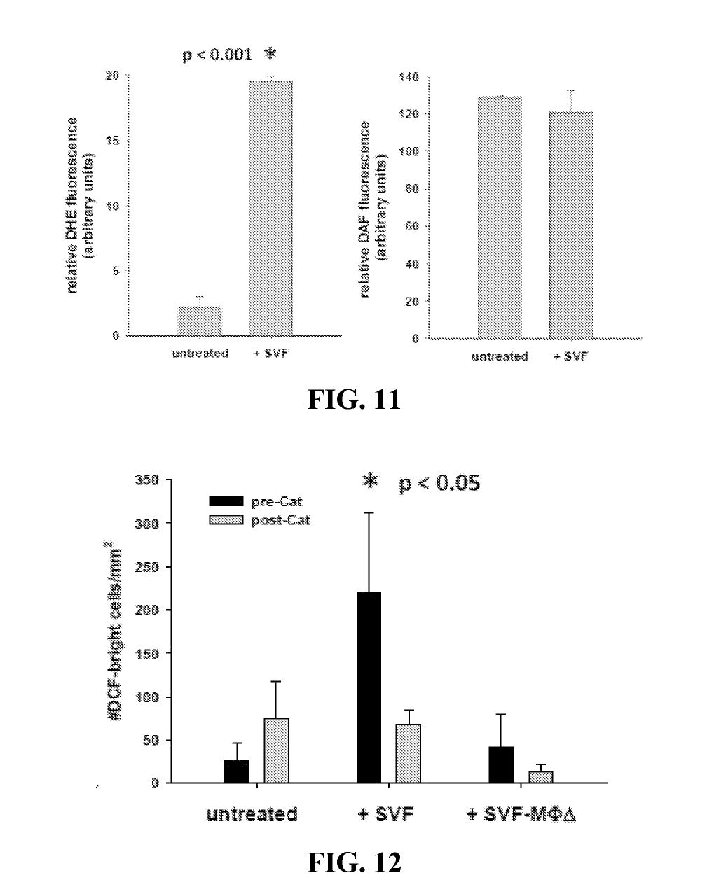

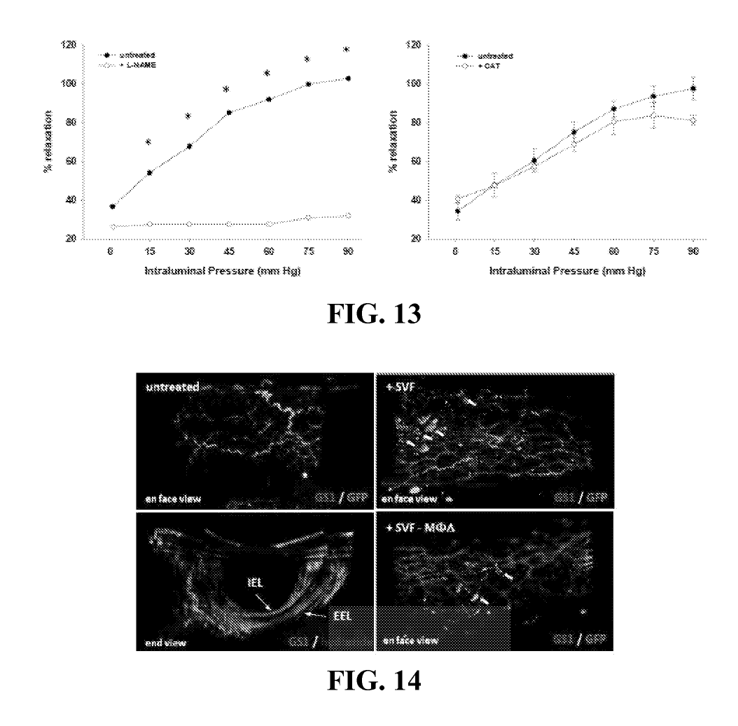

Because there was no change in medial mass (FIG. 6, FIG. 7), it was assumed that the increased diameter was due to vasodilation, either constitutive or in response to the vasodilators used during fixation at explant. In isolated, pressurized vessel preparations, saphenous arteries from untreated, SVF cell-treated, and SVF-MΦΔ cell-treated normal mice exhibited comparable endothelium-dependent and -independent vasodilation responses (FIG. 8). However, vessel relaxation to sequentially increasing changes in intravascular pressure was significantly enhanced in saphenous arteries from mice treated with the SVF cells, but not the MΦ-depleted SVF cells (FIG. 8). These differences in diameters were not observed in the absence of extravascular Ca⁺², consistent with the absence of any structural remodeling (FIG. 8). The influence on pressure-dependent, but not agonist-dependent, relaxation is analogous to changes in myogenic tone in resistance vessels whereby reduced relaxation for a given pressure reflects reduced resting tension in the smooth muscle layers. While there are potentially many pathways mediating myogenic tone, one prevalent regulatory mechanism involves a balance of vessel wall reactive oxygen species (ROS). (Wagenfeld, et al., 2013) Hydrogen peroxide (H₂O₂), one such ROS, is a potent vasodilator (Matoba, et al, 2000) and the reduction in O₂⁻ anions associated with the conversion to H₂O₂ preserves nitric oxide thereby further promoting vessel relaxation. (Kang, et al, 2009) Using 2′,7′-dichlorofluorescein (DCF) to detect H₂O₂ and dihydroethidium (DHE) to detect O₂⁻, saphenous arteries isolated from mice treated with SVF cells exhibited higher H₂O₂ and O₂⁻ levels, but not NO levels, in the vessel wall as compared to untreated arteries (FIG. 9, FIG. 10, FIG. 11, FIG. 12). Importantly, H₂O₂ levels in MΦ-depleted SVF cell-treated vessels were not elevated (FIG. 9, FIG. 10). Scavenging H₂O₂ with tissue permeant PEGylated-catalase (Phillips, et al, 2007) (FIG. 10) in saphenous arteries from mice treated with the SVF cells eliminated the cell-dependent decrease in myogenic tone, mimicking the pressure responses of MΦ-depleted SVF cell treatment (FIG. 8). This is consistent with an adipose SVF dependent, macrophage (CD11b⁺)-derived, H₂O₂ mechanism of small artery relaxation super-imposed on the normal dependency on nitric oxide (NO) for baseline relaxation to myogenic constriction (FIG. 13).

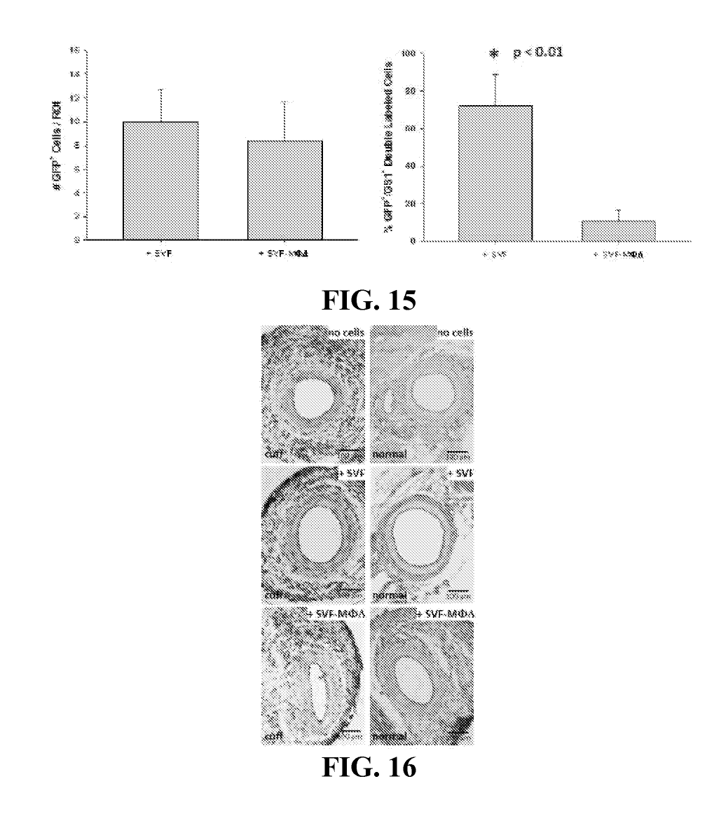

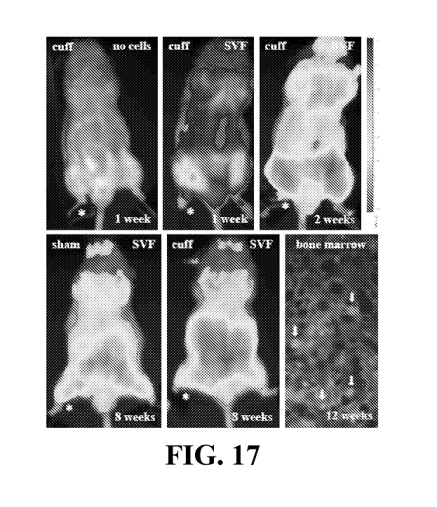

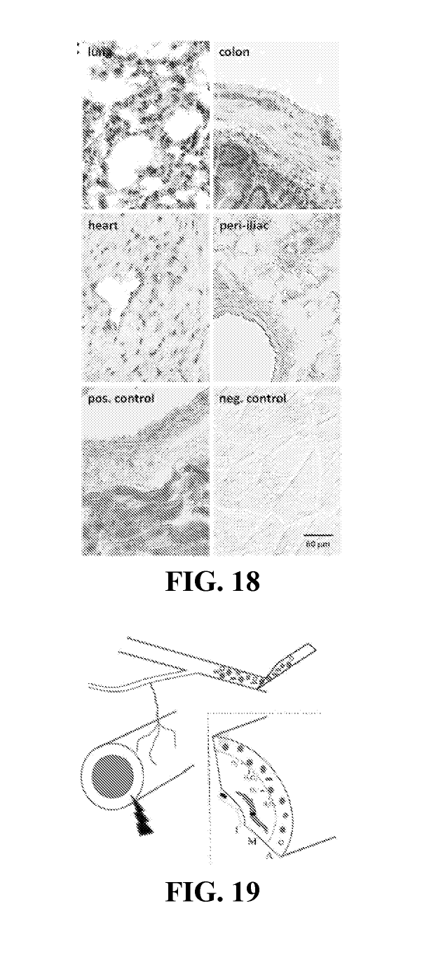

From the DCF fluorescence images, it appears that there were higher numbers of DCF-bright (i.e. H₂O₂-producing) cells in the walls of SVF cell-treated arteries than in untreated or SVF-MΦΔ-treated arteries (FIG. 9, FIG. 11), suggesting that additional cells have infiltrated the artery wall in the SVF treated mice. Indeed, further analysis revealed the presence of intravenous-delivered SVF cells within the adventitia, but not in the media, of saphenous arteries (FIG. 14, FIG. 15) (FIG. 16). The adventitia of saphenous arteries from mice treated with MΦ-depleted SVF isolates also contained cells from the isolate (FIG. 14, FIG. 15). However, significantly more of the infiltrated cells from the SVF cell isolate were positive for GS-1 lectin (a marker of both mouse endothelial cells and tissue macrophages (Maddox, et al, 1982; Laitinen, 1987)) than from the SVF-MΦΔ cell isolate (FIG. 14, FIG. 15). The infiltration of the artery wall most likely occurred via the vaso vasorum of the adventitia, suggesting that the SVF cells may have distributed throughout the body of the mouse following intravenous injection via the vascular supply. Indeed, SVF cells were found distributed throughout the mouse, including in the bone marrow, by one week and as late as three months post-injection, (FIG. 17). Luciferase-positive cells from the SVF isolate were present within a variety of tissues throughout the animal following intravenous injection (FIG. 18).

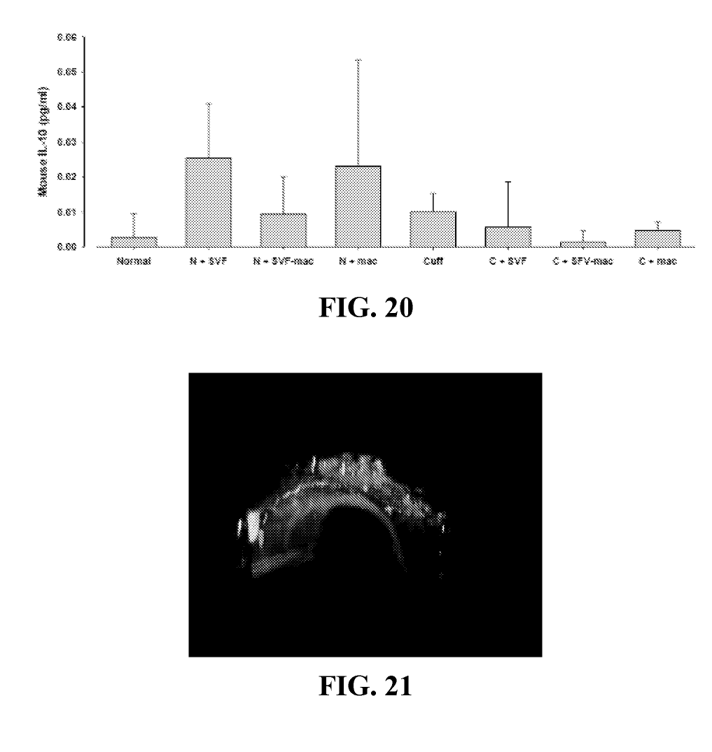

Based on the findings, a mechanism of adipose SVF cell-mediated vessel tone control in which CD11b⁺ cells normally resident within lean, white adipose spontaneously populate the vessel wall and generate H₂O₂ leading to reduced myogenic tone (FIG. 19) is proposed. Because these cells are residing within the adventitia of the vessels, it is likely that they infiltrated the vessel wall via the nutritive vaso vasorum as they circulated throughout the vasculature. As such, the injected cells should be capable of disseminating throughout the body entering any and all vascularized tissues. While not tested directly (for technical reasons related to the cuff), it is presumed that a similar mechanism is occurring in insulted vessels, as the saphenous artery embedded in the inflamed tissue of the cuff model exhibited vasodilation following SVF cell treatment. Regardless, this mechanism implies a local effect on vessel wall physiology. No significant changes in elevated plasma IL-10 levels in any of the cell treated mice (cuffed or normal, FIG. 20) were observed, suggesting that a systemic-wide impact is not present or is minimal. These findings also support a therapeutic strategy targeting the microvasculature, via resetting myogenic tone, as a clinical target for ischemia described by others. (Hill, et al., 2009; Palomares, et al., 2013) It is thought that key to this therapeutic goal is the favorable resetting of myogenic tone without changing neurohumoral regulation (Hill, et al., 2009), the very activity displayed by the SVF cells in these experiments.

Given that the relative absence of macrophages in the CD11b⁺-depleted SVF coordinated with the loss of tone relaxation and the absence of GS-1-positive cells within the vessel wall, it is presumed that the infiltrating cells from the intact SVF isolate are macrophages (and possibly even CD301⁺, M2 class, pro-healing macrophages). Additionally, macrophages express the hydrogen peroxide-producing enzyme super oxide dismutase-3 (SOD-3) externally on their cell surface (Fukai, et al., 2002), further implicating the macrophage as the relevant cell type in the SVF isolate. Whether these macrophages infiltrated the vessel wall as macrophages (already resident in the adipose at the time of harvest) or newly recruited monocytes present in the isolate is not clear. Furthermore, it is not clear if this pro-vascular activity is unique to white adipose-resident macrophages or why there is a high proportion of tissue protective, pro-healing macrophage subtypes in adipose (Murray, et al., 2011; Lumeng, et al., 2008). Interestingly, adult mice lacking CD11b⁺ cells become obese (Dong, et al., 1997) and the tissue-resident macrophages modulate immune homeostasis in adipose (Morris, et al., 2013) suggesting a role for macrophages in adiposity and adipose tissue homeostasis. Perhaps, in the SVF cell therapy, these homeostatic macrophages from adipose mimic the endogenous activity of macrophages normally resident within the artery wall, thereby enhancing normal vessel wall physiology.

Recent investigations into vascular-centered, cell-based therapies have focused on the use of mesenchymal or hematopoietic stem cell populations to promote angiogenesis and arteriogenesis. Unlike many of the typical regenerative cell preparations, the freshly-isolated adipose SVF preparation is predominately made up of tissue homeostasis cells with proportionally limited numbers of mesenchymal stem cells. The present disclosure provides a potential therapeutic activity of the adipose SVF cells, that is, distal vessel tone control, which compliments the varied therapeutic activities already ascribed to adipose-derived cells.

Given that most diseases have multiple and varied underlying complications and dysfunction, such a multi-pronged therapy opens up new avenues of therapeutic investigation. Combined with the very safe use of adipose-derived cells (13), the relatively easy harvest of high numbers of therapeutic cells without additional manipulation (including in point-of-care settings), and the autologous nature of the therapy, this therapy is poised to have a significant clinical impact on highly vascular-dependent disease conditions such as Reynaud's phenomena/scleroderma, ischemic deficits (e.g. MI and CLI), vasculitis, fibro-proliferative inflammatory conditions, hypertension, and diabetes.

### Example 2

It is suggested that, in certain embodiments, a subset of macrophages within the therapeutic cell preparation promotes vasodilation by selectively modulating vascular myogenic activity. To address this, a series of experiments designed to define macrophage therapeutic dynamics and determine the mechanism by which adipose-derived macrophages mediate myogenic responsiveness in arteries collected from cell-treated mice (including exploring reactive oxygen as a mechanistic mediator) is performed.

Dysfunction of the distal vasculature causes or complicates many human diseases/conditions including, for example, hypertension, diabetes, peripheral vascular disease, and ischemia. An effective therapy will target the many dysfunctional vessels distributed throughout the distal vasculature and normalize vasoactive responses. Using mouse models of small artery vasculitis and vasoactivity, a sub-population of adipose tissue-resident cells that promote controlled vasodilation of distal, small arteries via modulation of vascular myogenic tone is identified. Moreover, it is suggested that a tissue-resident, macrophage in the heterogeneous adipose stromal cells can populate small artery walls and positively mediate myogenic tone control. This therapy may have broad clinical utility. Furthermore, this work will provide new insights into vessel-stroma biology as the proposed cells mediating tone control constitute normal tissue-homeostatic cell populations.

Define the macrophage-dependent mechanism underlying the pro-vasodilation activity of therapeutic stromal cells. A variety of vascular and stromal cell types, including resident immune cells, have been identified within the adipose-derived cell isolate. Removal of over 80% of the macrophages (via CD14⁺ cell depletion) from the total isolate attenuates the pro-vascular activity of the total cell isolate. Based on this finding, additional preliminary evidence, and the diversity of adipose macrophage phenotypes, it is suggested that a subtype of adipose tissue-resident macrophage is necessary for the pro-vascular activity. Accordingly, different strategies of select cell deletion (magnetic cell sorting and diphtheria toxin receptor transgenics) are used to identify the specific sub-type(s) of macrophage in the therapeutic cell isolate mediating vasodilation in normal and injured animals (vasculitis and ischemic muscle conditions in mouse). Specific cell populations within each sub-fraction are characterized using cell markers and functional assays. Fluorescence reporter mice serve as the source of tissue cells and/or assay animals to facilitate cell tracking.

Determine the ROS-dependent mechanism by which therapeutic stromal cells mediate myogenic tone. Findings from isolated vessel experiments indicate that treatment with the therapeutic cells relaxes myogenic tone but do not alter responses to vasodilating agents. Changes in reactive oxygen species (ROS) within the vessel wall significantly impact myogenic tone. Moreover, there is evidence that scavenging H₂O₂ in small arteries from mice treated with the adipose therapeutic cells eliminates the cell-dependent reduction in myogenic tone, suggesting that ROS dynamics in the walls of cell-treated vessels drives the cell-dependent change in myogenic tone. The isolated vessel preparation and indicator dyes will continue to be used to assess relative O₂⁻ and H₂O₂ levels in combination with cell depletion approaches to determine cell-specific ROS modulation. Furthermore, select anti-oxidants are used to evaluate changes in myogenic tone of adipose cell treated vessels. Prompted by the presence of therapeutic cells within the vessel wall, the live isolated vessel preparation is combined with confocal microscopy and reporter transgenics to simultaneously locate therapeutic cell subtypes and measure vasoactivity with ROS perturbations.

Effective tissue perfusion is the primary function of the distal vasculature. Too little blood flow leads to ischemia and tissue dysfunction. Too much leads to complications related to increased pressures such as edema and capillary rarefaction. Moreover, loss of proper flow control contributes secondarily to long-term, pathological adaptations to the cardiovascular system (e.g. negative vascular remodeling). While vasoactivity is regulated via a number of pathways, it generally involves lessened or reversal of vascular smooth muscle contraction. Normally, the vessel is in a partially contracted state exhibiting some level of “tone”, established through a variety of mechanisms extrinsic and intrinsic to the vessel wall. A core intrinsic mechanism establishing tone involves the myogenic response to changes in intravascular pressure (sensed, in part, by the smooth muscle as stretch) whereby concentric constriction occurs as intravascular pressure increases. Endothelial cell influence is superimposed on this baseline level of myogenic constriction (myogenic tone is established even in the absence of the endothelium) (17, 18). Therefore, any changes to vessel diameter occur relative to this steady-state, tone-dependent diameter. Given this importance, manipulating the myogenic status of the distal vasculature could be the basis of a general therapeutic strategy intended to reduce peripheral vascular resistance and improve perfusion, which is often compromised in many diseases.

Given the complexities inherent in vascular functional deficits, an ideal therapy would be one that could dynamically address only the vascular dysfunction: treating all of the affected vessels while not influencing un-affected vessels elsewhere in the vascular tree. Furthermore, such a therapy should not over-ride other, homeostatic mechanisms. For example, common clinical strategies to address distal vessel dysfunction, particularly following an ischemic attack, involve systemic administration of pharmacological dilators targeting relaxation of vascular smooth muscle (19-28). While effective at dilating vessels, such agents act on all responsive vessels (whether desired or not) and can “swamp out” other vasoactive signals.

Given the appropriate cell type, a cell-based therapy could provide a viable solution. While drugs, in general, target one underlying imbalance, a therapeutically capable cell (or cell mix) has the potential to address many imbalances in an adaptive manner; cells can respond differently as inputs change. With respect to vessels, there is a rapidly growing appreciation for the role of a variety of cell types resident within the vessel wall in establishing vessel homeostasis and, coordinately, normal vascular function (29, 30). This cellular dynamic, which includes the activities of mesenchymal stem cells, immune cells, and fibroblast phenotypes, could be mimicked or leveraged to re-establish normal vessel function in distal vessels.

Adipose-derived stromal vascular fraction (SVF) cells are increasingly being explored as a cell-based therapy for a number of disease conditions including chronic tissue ischemia, autoimmune disorders, and tissue repair (31-36). Typically derived via enzymatic digestion of subcutaneous lipo-aspirates followed by removal of the adipocytes, the harvested stromal vascular fraction contains a heterogeneous mix of cells including endothelial cells, perivascular cells (e.g. smooth muscle cells, pericytes), fibroblasts, and multi-potent cells (32, 37-41). The SVF also contains resident immune cells such as regulatory and natural killer T-lymphocytes, B lymphocytes, dendritic cells, and macrophages (32, 37-41).

Most research related to vascular-centered, cell-based therapies has focused on the use of mesenchymal or hematopoietic stem cell populations to promote angiogenesis and arteriogenesis. However, a new potential therapeutic activity, distal vessel tone control, by a mixed population of cells freshly derived from adipose that are predominately made up of tissue homeostasis cells (with limited numbers of mesenchymal stem cells) is proposed by the present disclosure. This new, pro-vascular activity compliments the varied therapeutic activities already ascribed to adipose-derived cells: pro-angiogenic, anti-inflammatory, and pro-healing. Given that most diseases have multiple and varied underlying complications and dysfunction, such a multi-pronged therapy, which includes, for example, normalization of flow control, opens up new avenues of investigation for therapies.

The present disclosure provides an understanding of post-ischemia vascular dysfunction and explores cell-based therapies for such conditions.

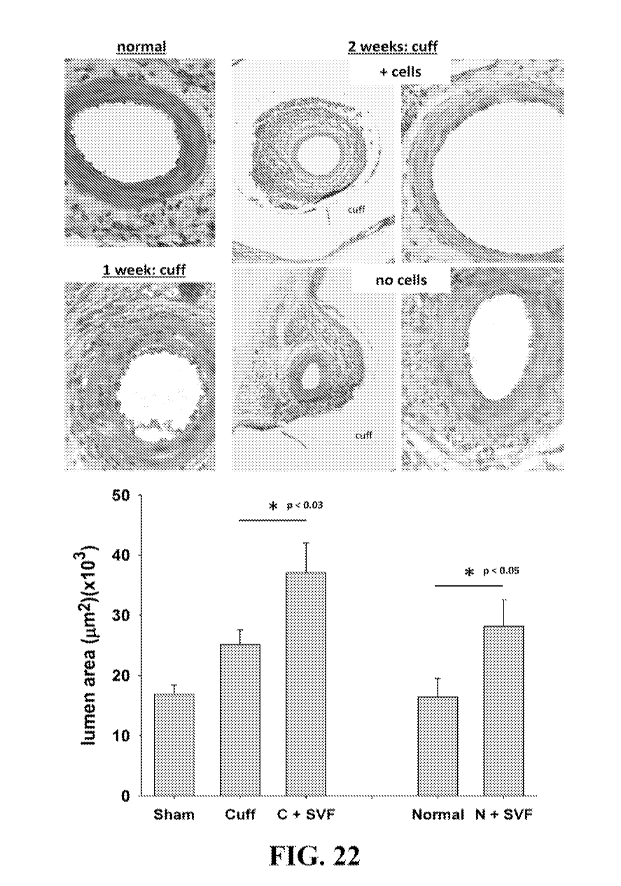

In one line of investigation, it was found that despite a sufficient vascular density, there is considerable dysfunction in the distal vessels of the repaired hind limb (45, 46, 55). In a combined effort to develop a therapy targeting this dysfunction, a set of experiments were performed to assess the role and efficacy of tissue stromal cells in regulating vascular function. Because it was desirable to localize an insult to a single vascular segment in these particular experiments, model of local vascular injury (56, 57) was adapted. In these experiments, the right medial saphenous artery in a mouse was loosely cuffed with a short piece of PE tubing to induce a materials-driven inflammation which leads to vascular insult. After a week with the cuff in place, each mouse was administered, via the tail vein, 1×10⁶ cells (the stromal vascular fraction, SVF, of adipose tissue) harvested from syngeneic donor reporter mice constitutively expressing luciferase and GFP. One week later, mice were perfusion-fixed under constant (100 mm Hg) pressure in the presence of a vasodilator cocktail and the vessels explanted. The diameters of the cuffed vessels were considerably larger when the mice were treated with the SVF cells (FIG. 22). Surprisingly, arteries from normal, non-injured mice treated with cells were also larger in diameter.

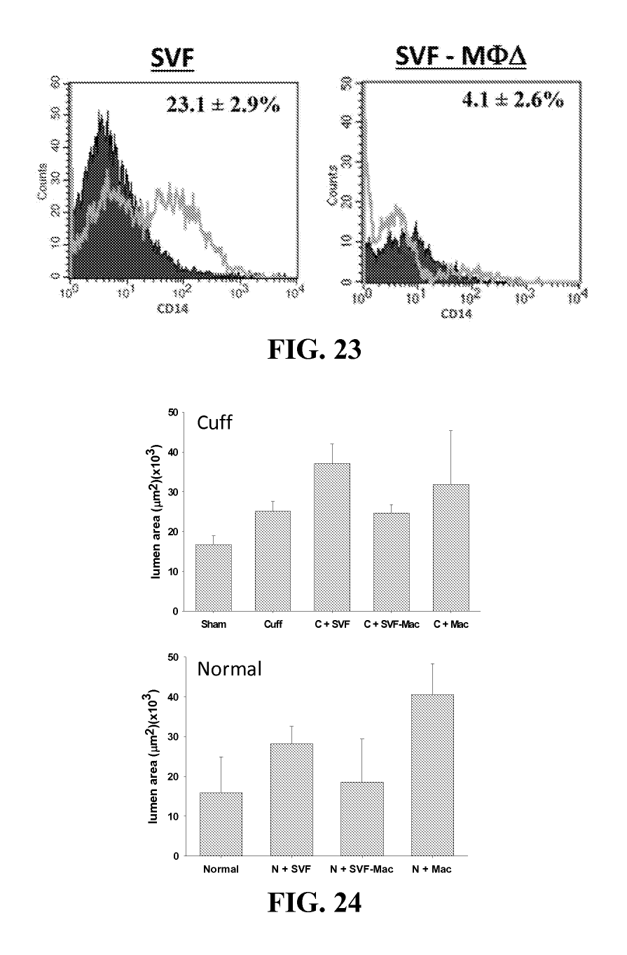

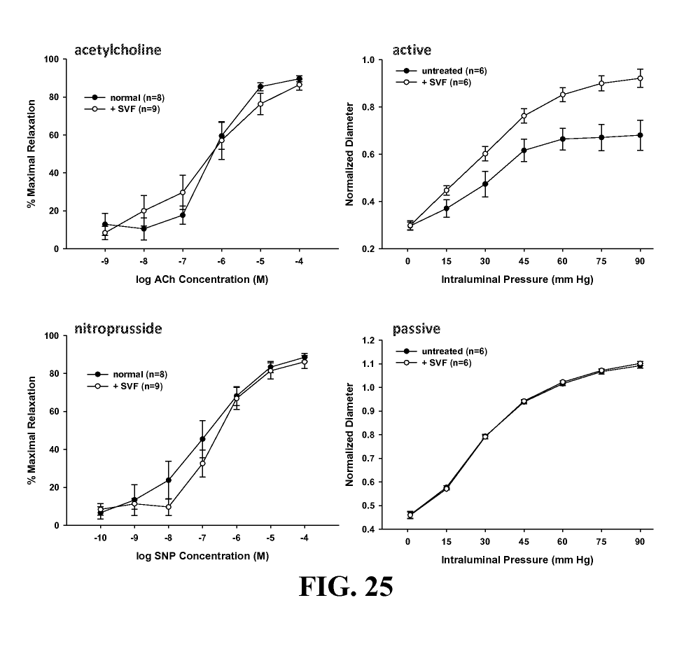

The fresh cell isolate delivered to the mice was a mixed population of cells derived from the microvasculature and stroma of adipose tissue. Macrophages (MΦs) are known to establish tissue homeostasis (41, 58), are prevalent in adipose tissue (59), and represent a relatively large fraction of cell types present with the isolate (data not shown). Consequently an experiment was performed to determine if macrophages are responsible for the vasodilation. Magnetic beads coupled to CD11b antibodies were used, to deplete the isolate of MΦs (CD14+ cells) prior to intravenous injection into mice (FIG. 23). As suspected, depletion of the MΦs from the isolate attenuated the vasodilation activity of the total cell isolate in both cuffed and normal mice (FIG. 24). Interestingly, delivery of just the macrophage-enriched sub-fraction (i.e. only the collected CD11b⁺ cell population) promoted vasodilation to a similar (cuffed) or greater (normal) extent as the total isolate (FIG. 24). These depletion experiments suggest that MΦs are necessary for the pro-dilation activity of the vascular stromal cell preparation.

The changes in saphenous artery diameter observed in cell-treated mice suggest that the cells promoted either vasodilation or a physical outward remodeling of the vessel wall. Because these changes in diameters were not accompanied by changes in wall mass (medial areas were not affected by cell treatment, data not shown), it is presumed that vasodilation was the underlying cause. To test this, saphenous artery segments are isolated from normal untreated and cell-treated mice and evaluated vasoactive responses to a variety of stimuli in a pressurized isolated vessel system (60). In these experiments, the segments were pre-constricted with phenylephrine to approximately 60% of resting diameters at 50 mm Hg. Vasodilation responses to 5 log-order doses of acetylcholine or sodium nitroprusside were not different between cell-treated and untreated vessels (FIG. 25). However, the artery segments from cell-treated mice were significantly more dilated with increasing intraluminal pressures (FIG. 25). These differences in diameter were not observed in the absence of extravascular Ca⁺² indicating that the vessel wall had not been structurally remodeled (FIG. 25). Interestingly, the cell-treated vessels exhibited proportionally larger diameters at intraluminal pressures comparable to that normally experienced by the mouse femoral artery in situ (˜50 mm Hg (61)), consistent with the histology results.

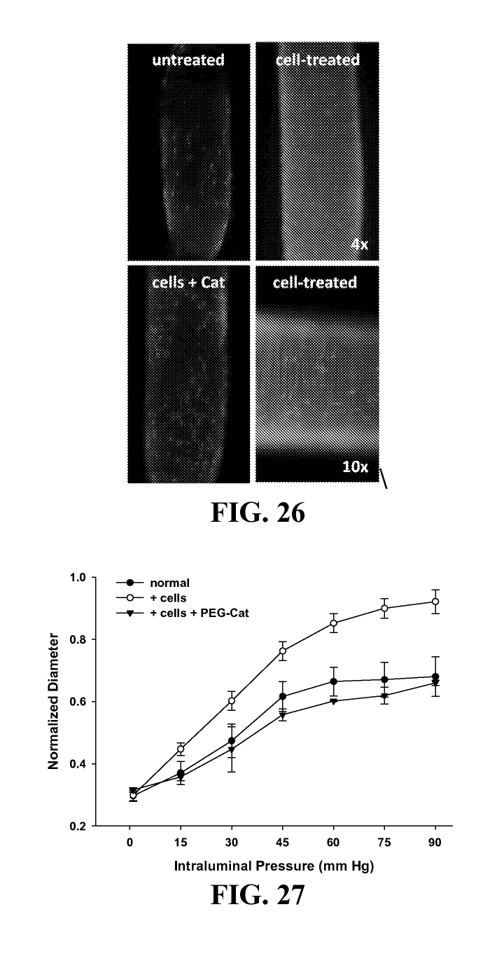

The relaxation of cell-treated vessels in response to increasing intraluminal pressures, without a change in responsiveness to nitric oxide-induced vasodilation, suggests that the injected cells have specifically altered the myogenic responsiveness of the vessels. The absence of vessel wall remodeling indicates that this change in myogenic response is not due to any change in smooth muscle mass or wall extracellular matrices. While there are a potentially many pathways mediating myogenic tone, a prevalent one involves a balance of vessel wall reactive oxygen species (ROS), particularly the conversion of superoxide anion to H₂O₂ via superoxide dismutase (62-65). This pathway is even more appealing given that H₂O₂ is a potent vasodilator (66). Also, the reduction in O₂ anions preserves nitric oxide thereby further promoting vessel relaxation (67). Finally, MΦs (the evidence indicates is necessary for the vasodilation activity) constitutively express extracellular superoxide dismutase (EC-SOD), an enzyme involved in converting O₂⁻ to H₂O₂ (68-69). Thus, generation of H₂O₂ via the cell treatment would explain the observations. Using 2′,7′-dichlorofluorescein (DCF) to detect H₂O₂, it was observed that H₂O₂ levels in the vessel wall are elevated in saphenous arteries treated with non-GFP tagged SVF cells as compared to untreated arteries (FIG. 26). Interestingly, there appears to be more DCF-positive cells in the cell-treated vessels than in the untreated vessels. Importantly, scavenging H₂O₂ with tissue permeant PEGylated-catalase (70) in saphenous arteries from mice treated with the total cell isolate eliminated the cell-dependent decrease in myogenic tone (FIG. 27), consistent with an SVF cell-derived, H₂O₂ mechanism of relaxation (PEG-catalase won't enter cells resulting in them remaining DCF-bright). Based on this evidence and the current thinking on the mechanisms of vessel tone control, it was hypothesized that in the cell-treated mice, H₂O₂ is generated in the vessel wall via superoxide dismutase expressed by the delivered MΦs populating the vessel wall. It is this elevated level of H₂O₂ that leads, directly or indirectly, to a reduced myogenic tone.

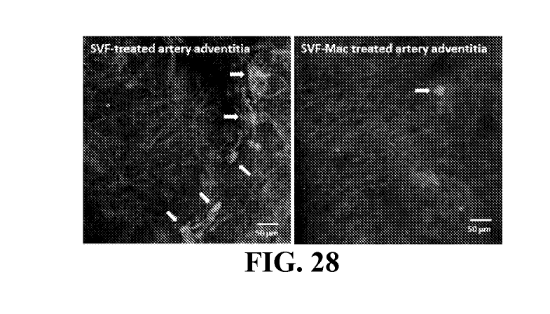

The isolated vessel preparation indicates that the pro-relaxation phenotype persists even with the removal of the vessel from systemic stimuli, consistent with the idea that the active therapeutic agent(s) (i.e. cells) are present within the vessel wall. As stated, it is suggested that the relevant populating cell type is the MΦ. Indeed, tracking of the injected cells (via the transgenic expression of GFP) indicates that cells of the total isolate do indeed populate the adventitia of normal arteries, the number of which is attenuated following MΦ depletion (FIG. 28).

### Research Design and Methods

Findings indicate that adipose-derived therapeutic cells modulate small artery myogenic tone in a macrophage- and ROS-dependent manner. Moreover, this effect is lasting (measurements were made 1 week after a single bolus intravenous injection of cells) and is maintained even when the artery is isolated free from the animal. The proposed work tests the overall hypothesis and will identify cellular and molecular mechanisms of action by determining the dynamics of M□s in the relevant vasoactive effect and defining vessel wall reactive oxygen status (important in vessel tone regulation).

Aim 1: Define the macrophage-dependent mechanism underlying the pro-vasodilation activity of therapeutic stromal cells. The depletion experiments suggest that MΦs are necessary for the pro-dilation activity of the vascular stromal cell preparation. This suggestion raises a number of interesting questions that will be addressed in this Aim which are intended to refine the existing understanding of the role of the macrophage as the mechanistic cell-type of vasodilatory action.

Defining the cell phenotypes present within the macrophage-enriched, CD11b⁺ fraction The depletion experiments involve targeting CD11b to capture and remove macrophages/monocytes. While CD11b is expressed by all tissue macrophages (71) and is commonly used as a marker of macrophages/monocytes (72), other blood-borne cell types also express CD11b, confounding the depletion study findings. It has been confirmed that MΦs are depleted from the total cell isolate using a more specific MΦ marker (CD14). Yet, it still needs to be determined what, if any, other cell types are also depleted by the CD11b-based approach and if these cell types are contributing to the pro-dilatory activity. Furthermore, it is becoming clear that macrophages display a continuum of phenotypes ranging from pro-inflammatory to trophic/homeostatic (41, 59, 73). It would be important to determine which MΦ sub-type is present within the isolate, particularly given the dynamic phenotypic modulation of MΦs within adipose (59).

Experimental Design The first step is to define the cell composition of the removed CD11b⁺ fraction. To do this, flow cytometry using markers commonly used to identify those cell types known to express CD11b is employed. These include lymphocytes (CD2⁺), mast cells (FεR1⁺), neutrophils (Ly6G⁺), dendritic cells (CD80⁺) and MSCs (CD105⁺). The F4/80 marker is also used as an alternate, confirmatory marker for MΦs (distinct from monocytes—addressed further in Aim 2). In these experiments, the therapeutic cell total isolate is derived from FVB/n tie2:GFP transgenic mice (syngeneic with the cells used in the aforementioned experiments) to take advantage of the endogenous GFP expression marking endothelial cells (74) to assess endothelial cell contaminants (CD31, the typical EC marker, is cleaved by the enzymes used to harvest the isolate). As before, the total isolate is collected and then part of the isolate is depleted of CD11b⁺ cells with the magnetic separation system (Miltenyi). All three resulting fractions, the total isolate, the depleted isolate (lacking CD11b⁺ cells), and the removed fraction (enriched with CD11b⁺ cells) will be analyzed using an 8 channel flow cytometer (BD LSRII), which permits analyses of marker combinations to more effectively identify cell types. In a separate set of experiments, isolates/fractions in which the cells were derived from FVB/n CD11b-DTR-eGFP transgenic mice (72, 75) will be analyzed. In this mouse, the human diphtheria toxin receptor and GFP transgenes are expressed under the control of the CD11b promoter. This dual transgene cassette permits tracking of CD11b⁺ cells via GFP expression as well as the selective deletion of CD11b⁺ in the mouse following IP delivery of diphtheria toxin via only the human DTR (72, 76). To selectively kill the CD11b⁺ cells prior to cytometry, the donor mice will be given diphtheria toxin 4 and 2 days prior to adipose and cell harvest. The CD11b-DTR-EGFP mouse is available from Jax labs, and a colony will be established therefrom.

Finally, CD11b⁺ macrophages will be characterized further to determine the spectrum of phenotypes present in the isolate. Others examining adipose-resident macrophages have broadly categorized macrophages as either M1 (pro-inflammatory) or M2 (pro-homeostatic) (59, 77). Because an underlying assumption is that the pro-dilatory macrophage in the therapeutic isolate contributes to homeostasis (and not necessarily inflammation), it is worthwhile to perform this additional level of characterization. To do this, cells isolated from adipose harvested from CD11b-DTR-eGFP transgenic mice will be used to endogenously tag CD11b macrophages. In cell smears, immunostain for M1 (iNOS, CD11c, and CXCL10) or M2 (arginase, MGL1, CD206, and CCL22) types co-localizing each to GFP⁺ cells (F4/80 immunostaining to verify the macrophage identity) will be used. In this way, the relative percentage of M1 and M2 macrophages in the isolate can be determined. To validate this approach, adipose from lean (i.e. normally fed) and obese (high fat fed) mice will be used, which switch from predominately an M2 phenotype in lean adipose to a predominately M1 phenotype in obese mice (40).

It is expected that MΦs (and monocytes) constitute the majority of cell types within the CD11b⁺ isolate. Indeed, in a quick preliminary experiment, greater than 50% of the CD11b⁺ fraction is CD14⁺ (with the remaining cell types being predominately endothelial cells non-specifically trapped within the column as small microvessel fragments resulting from incomplete adipose digestion). Should it be found that there is another prevalent cell type (e.g. represented at >10%), the saphenous artery dilation experiments in which the specific cell type has been depleted will be repeated to determine the involvement of this alternate cell type. CD11b may not mark all MΦs. However, experiments show that removal of MΦs (CD14⁺ cells) expressing CD11b effectively removes the pro-dilatory activity of the isolate. Thus, while all MΦ types have not been removed, it appears that those expressing CD11b are the relevant MΦs. For the cytometry, commonly used cell markers are chosen. Additional markers can be employed as needed. While primarily descriptive, the experiments in this section better define the isolates and sub-fractions.

Tracking of Macrophages Cell fractions are systemically delivered into mice via a tail vein injection. Also, the resulting dilation of the saphenous artery persists for at least a week following the cell injection. Combined, these findings suggest either a lasting change in vessel wall phenotype or that the pro-dilation cells take up residence within the vessel wall and continuously effect vessel tone. Indeed, injected cells within the wall of the dilated saphenous artery (as well throughout the mouse including the bone marrow—see FIG. 27) are observed, inferring that the latter situation is occurring. But, it has not yet been determined whether these infiltrating therapeutic cells are in fact the MΦs. Therefore, a series of experiments is performed to determine where (within the vessel wall and other tissues) and for how long MΦs in the therapeutic isolate distribute following intravenous injection.

Experimental Design Throughout these experiments, the CD11b-DTR-eGFP transgenic mouse is used as the therapeutic cell source, relying on the CD11b-driven GFP expression as a reporter system and normal mice as the recipients. Additionally, as with all of the delivery experiments of this entire project, 1×10⁶ cells are injected per mouse. In the first round of experiments, MΦs will be located, via the GFP fluorescence, in fixed-frozen sections (to avoid paraffin processing) of explanted tissue. Furthermore, saphenous artery segments will be isolated and examined via confocal microscopy to localize M□s. Subsequent image analysis, including 3D rendering of the image stacks, will be used to identify the location of GFP-positive cells within the different layers of the vessel wall. Confocal microscopy is used in order to image properly through the vessel wall (which is approximately 50 μm in thickness). Alexa 644-hydrazide is used to identify the internal and external elastic laminae (78), thereby defining intima, media, and adventitia layers. Similar approaches will be used to locate MΦs elsewhere in the mouse including other vessels, other tissue beds, and bone marrow.

In the second round, diphtheria toxin (25 ng/g) is used to selectively deplete MΦs in the total isolate and in treated mice. As before, vessel morphology and cell locations in sections and whole isolated segments is assessed by confocal microscopy. In these experiments, toxin will be: (i) administered to donor mice four and two days prior to cell harvest; (ii) pre-incubated and co-delivered with the isolated cells to recipient mice; or (iii) administered three days and five days after delivery to recipient mice. All treatment groups will be explanted 7 days after delivery. DT administrations 1 and 2 are designed to deplete source MΦs (while accounting for potential changes in cell population distributions within the source adipose). DT administration 3 will delete macrophages once the donor cells have already taken up residence. Because the recipient mouse cells do not express the DT receptor, only the therapeutic MΦs will be affected. Cells expressing the human DT receptor begin to die within hours of exposure to this dose of DT (75), with the DT-dependent absence of MΦs persisting for 48 hours in intact mice (75). A non-biologically active form of DT will be used in a subset of experiments as a negative control.

In the third round, the CD11b-DTR-eGFP transgenic mice will be used as recipient mice (as opposed to cell donor mice will be WT) to track the behavior of endogenous MΦs in the pro-dilation activity. Here, therapeutic cells will be isolated from normal, non-reporter mice followed by standard analyses. Experiments will be also conducted in which the CD11b-DTR-GFP recipient mouse is depleted of endogenous macrophages prior to injection of wild type cells using again the DT dosing regimen of four and two days prior to cell delivery. Others have described the presence of resident MΦs within the adventitia of mouse arteries (30).

When possible, F4/80 fluorescence-immunostaining co-localization with GFP will be used to distinguish macrophages from monocytes in all experiments. Finally, the first round of experiments will be repeated using tie2:GFP transgenic mice as the source of isolates to track endothelial cells as an example mesenchymal cell as a contrast to MΦs and to demonstrate cell-type specificity. While mesenchymal stem cells also represent a good alternate candidate (given that they too can home to specific tissue sites), endothelial cells represent 30+% of the total isolate and some are inadvertently pull down with the bead depletion process (data not shown).

While not wishing to be bound by theory, it is believed that these experiments will find GFP⁺ macrophages populating vessel walls (and likely other tissues throughout). It is suggested that the MΦs are entering the vessel wall via the vaso vasorum of the adventitia. Coupled with the confocal imaging and the hydrazide staining, it can be determined if the cells are within the adventitia or throughout the wall layers. If needed (i.e. cells are observed in all layers), a time-course experiment can be performed to map the progressive re-population of the vessel wall. In the depletion experiments, an absence of CD11b-GFP⁺ cells is predicted to be present within the vessel wall. Others have shown a reduction in macrophage levels down to ˜5% (75) with the diphtheria toxin treatment. While this is not a complete elimination of macrophages, it is similar to the bead deletion approach which demonstrates an effect. It is not clear what to expect in the third round of experiments in which endogenous macrophages are to be depleted. While macrophages normally populate artery walls, there certainly is not the same degree of vessel relaxation without the additional therapeutic cells. It is very likely, though, that there will be an interesting dynamic in which vascular activities of endogenous and therapeutic macrophages are additive. It is possible that CD11b-GFP⁺ cells will not be found within the vessel wall in the first round (although, this is unlikely given the data). If this occurs, other cells types within the isolate will be explored, based on the idea that macrophages are involved by directing target cell types (which are well-defined) via activation to the vessel wall.

These experiments have used adipose as the source of vascular and stromal cells for many reasons. Because the parenchyma of adipose (the adipocytes) is buoyant, isolation of the vascular stromal fraction is easy as this fraction pellets and the adipocytes float following centrifugation of the tissue digestate. Also, it has been demonstrated that the considerable regenerative and therapeutic activities of adipose stromal vascular fraction cells related to angiogenesis, inflammation control, tissue repair, etc. (42, 44, 79-86). Macrophages reside in virtually all tissue beds, contributing to the stromal fraction of tissue cells, many of which acquire tissue-specific phenotypes (41, 58, 73, 87). Therefore, it will be determined if the pro-vascular activity by the cells represents a generic MΦ function or is unique to the adipose macrophage. Furthermore, it will be determined if pre-activation of the MΦ alters this therapeutic vascular outcome.

Experimental Design To perform these experiments, MΦs will be collected from different tissue beds of CD11b-DTR-GFP mice (using either CD11b magnetic bead purification or standard MΦ harvesting methods), characterized via cytometry, delivered to syngeneic normal mice, and the saphenous artery will be analyzed for vasodilation. Initially, the spleen and peritoneal macrophages collected will be examined by standard methods involving adherence protocols. These resting MΦs will be delivered to mice via the tail vein as is or following activation (i.e. with LPS). In a second round, these experiments will be repeated with MΦs collected from lung, which has an active macrophage community (76, 88). Finally, as mentioned previously, there is a shift in M1 vs. M2 macrophage phenotypes in adipose depending on whether the adipose is harvested from obese or lean mice. To determine if the pro-vascular activity of adipose MΦs reflects either an M1 or M2 phenotype-specific behavior, the standard vascular protocol will be repeated using cell isolates derived from adipose harvested from lean or obese (diet-induced) mice.

It is not immediately apparent whether the pro-vascular activity by the adipose macrophages reflects a generic or adipose-specific MΦ activity. It is suggested that the therapeutic outcome in the experiments are due more to an “enrichment” for these pro-vascular cells within the recipient mouse rather than an activity specific to adipose MΦs. However, given the broad phenotypic variations displayed by MΦs of different tissue beds, it's possible that there may be unique capabilities within each tissue-specific population of MΦs.

Aim 2: Determine the ROS-dependent mechanism by which therapeutic cells mediate myogenic tone. Based on this preliminary evidence and the current thinking on the mechanisms of vessel tone control, it is hypothesized that in the cell-treated mice, H₂O₂ is generated in the vessel wall via superoxide dismutase expressed by the delivered MΦs populating the vessel wall. It is this elevated level of H₂O₂ that leads, directly or indirectly, to lessened myogenic tone.

Changes in reactive oxygen species (ROS) mediate relaxed tone: To address the dynamics of H₂O₂ in the cell-treated vessel wall, a number of aspects relevant to ROS and vessel relaxation will be evaluated. It is predicted that superoxide anions are converted to H₂O₂ via elevated superoxide dismutase (specifically EC-SOD or SOD3) activity. The increased production of H₂O₂ then acts directly to relax the vessel wall and/or indirectly by scavenging O₂⁻ and thereby preserving nitric oxide (NO) which promotes relaxation ultimately via the reduction of myosin light chain phosphorylation.

Experimental Design Experiments will assess relative levels of 1) H₂O₂ in the vessel wall, 2) scavenging H₂O₂, and 3) blocking NO production.

In the first set of experiments, the dihydroethidium (DHE) and 2′,7′-dichlorofluorescein (DCF) dyes will be used to assess relative O₂⁻ and H₂O₂ levels, respectively (60, 89-91) in isolated vessels collected from untreated or cell-treated (total isolate or MΦ-depleted) mice to assess relative levels of ROS. Isolated vessels will be incubated with dye prior to executing the myogenic protocol (progressive increases in intraluminal pressure). Appropriate fluorescence images will be collected before and after the myogenic protocol with fresh dye being added each time. ROS levels will be assessed by comparing fluorescence intensities captured with the same camera settings between the different experimental groups.

In the second set of experiments, select agents, diethyldithiocarbamate (an SOD inhibitor (92, 93)) and PEG-catalase will be used in the bath of these dye-loaded vessels to perturb ROS levels pharmacologically and evaluate changes in myogenic tone with and without cell-treatment. This will test the sensitivity of the dyes and determine that H₂O₂ is mediating vessel relaxation in this setting.

In the third round of experiments, inhibitors of nitric oxide synthase (L-NAME) will be included to assess the contribution of nitric oxide in the cell-mediated vessel relaxation of untreated and cell-treated normal vessels. To complement to the functional vasoactivity measurements in the hanging vessel system, the levels of myosin light chain (MLC) phosphorylation, a molecular indicator of tone status (94) will be concomitantly measured.

While not wishing to be bound by theory, it is suggested that H₂O₂ levels should be increased concomitantly with reduced O₂⁻ levels in the cell-treated vessels. Furthermore, scavenging of H₂O₂ should prevent the therapeutic myogenic relaxation observed and not have an impact on vessels treated with MΦ-depleted cells. Finally, blocking of nitric oxide production should not affect the cell-induced change in myogenic tone if the cell-derived H₂O₂ is acting directly on the vessel wall (i.e. as EHRF) and not secondarily through NO preservation. Of course, both direct and indirect avenues of regulation may occur coordinately. It is anticipated that there will be quantitative and not qualitative consequences to the different pharmacological and dye treatments due to less-than-perfect specificities and sensitivities of reagents. However, others have used these reagents and successfully detected significant differences in vasoactivity and ROS levels.

Therapeutic cell-dependent superoxide dismutase activity: The hypothesis-derived prediction that H₂O₂ is elevated in cell-treated vessels leading to relaxation is being addressed in the preceding experimental set. In this set, the prediction that the SOD expressed by the therapeutic cells and its activity is responsible for the observed cell-induced vessel relaxation will be tested. It the hypothesis, therapeutic cells reduce O₂⁻ via conversion to H₂O₂ within the vessel wall. Superoxide dismutase (SOD) is a central player in converting O₂⁻ into H₂O₂. Consequently, in this context, it is predicted that the therapeutic cells serve as a source of new SOD activity. There are three relevant SOD isoforms, SOD1 (CuZn-SOD), SOD2 (Mn-SOD), and SOD3 (EC-SOD) expressed at different levels within different cells types (62). However, in MΦs, the predominant isoform present is the extracellular or EC-SOD (SOD3) isoform (68, 95) with the other major SOD isoform, SOD1, being expressed progressively less as the MΦ differentiates (69). Because the evidence indicates that MΦs are necessary for vessel dilation, SOD3 is the focus in these experiments.

Experimental Design To do these experiments, cell isolates collected from SOD3 knockout mice will be used, and the same tone and ROS assessments will be performed on vessels isolated from untreated and cell-treated normal mice. Additionally, delivery of only the CD11b⁺ cell fraction from the SOD3^{-/-} isolate will assess macrophage-dependent SOD3 activity in the relaxation response. If resources permit, knockout mice for SOD1 will also be used as a cell source in a similar fashion to determine the specificity of SOD isoforms in the response (the SOD1 and 3 KO mice are available from Jax labs and are viable as adults (92, 96)). Coordinately, the expression of SOD enzymes and catalase within the different cell isolates and fractions (e.g. the CD11b⁺ fraction) will be measured. In a second set of experiments, mice that lack SOD3 specifically in MΦs will be created by crossing CD11b-Cre mice (97) with a floxed SOD3 mouse (95). In this way, SOD3 can be depleted specifically in CD11b⁺ cells, thereby preserving the ability of other cells to convert O₂⁻ to H₂O₂. In this regard, it can be detected whether SOD3 outside of the MΦ population is contributing in any way to the therapeutic vessel relaxation, an observation counter to the hypothesis. Importantly, in all cases, cytometry will be performed to determine any changes in the proportional distribution of cell types in the adipose isolate that might have occurred secondary to the loss of the SOD genes. As before, vasoactivity and ROS levels will be measured in the isolated vessels from untreated and cell-treated mice. In these experiments, wild type littermates derived from het×het breedings will serve as syngeneic recipients.

While not wishing to be bound by theory, it is expected that the absence of SOD3, even in the presence of macrophages, will attenuate or eliminate the myogenic relaxation. Similarly, it is expected that removal of SOD3 in just MΦs (using the floxed SOD3 mouse) without the removal of MΦs will also eliminate the myogenic relaxation. Except for the conditional SOD3 mouse (which will be generated via breeding), all of the other mice are available as adults and can be used immediately. The finding by others that the absence of SOD3 in the knockout mice does not appreciably change leukocyte populations (84, 88) is important for these experiments and eliminates a potential confounding issue. It is desirable to determine a possible role for SOD2. However, SOD2 KO mice do not survive long into adulthood. Also, the expression level of SOD2 in MΦs is low. Notably, SOD present within the native cells of the vessel wall is not addressed in these experiments. However, it is easy enough to do simply by using the knockouts as the recipient mice for wild type therapeutic cells.

Vessel-resident, rather than systemic, macrophages are important MΦs (or monocytes) emigrate from the blood space into the vessel wall, likely via the vaso vasorum of the adventitia. The consensus opinion is that circulating monocytes (precursors to tissue macrophages) exist as two subpopulations: a pro-inflammatory monocyte and a homeostatic monocyte (58, 98). With the two populations, the pro-inflammatory monocyte uniquely uses CCR2, the MCP-1 receptor, to emigrate from the blood space into the tissue space, while the homeostatic monocytes use CX₃R1, the receptor for fractalkine (98, 99).

Experimental Design In Aim 1, the prediction that M□s from the injected cell isolate are populating the vessel wall of small arteries is tested. In these further experiments, the analysis is extended by delivering the cell isolate but impairing the ability of the macrophages/monocytes to enter into the vessel wall. In this way, therapeutic macrophages will be present in the mouse but not in the vessel wall. To do this, the isolated, vasoactive vessel preparation will be combined with confocal microscopy and use cell isolates from knockout/reporter transgenic mice expressing fluorescent proteins via either the CX₃CR1 or CCR2 promoters (100, 101). Importantly, both of these reporter mice are targeted null mutants with the fluorescent transgene serving to simultaneously disrupt the CX₃R1 (homeostatic monocytes/macrophages) and CCR2 (pro-inflammatory monocytes/macrophages) genes (100, 101). Thus, as heterozygotes, gene function and monocyte behavior are sufficiently preserved, yet monocytes are tagged. In homozygous mice, monocytes are tagged, but they lack a recruitment-mediating gene product. Both genetic configurations will be used in these experiments to tag the respective monocyte subpopulations to 1) preserve recruitment (heterozygous) or 2) knockout out their recruitment ability (homozygous). In these experiments, cells from these different mouse groups will be injected into normal mice using cells from the different reporter strains. In these experiments, wild type littermates derived from het×het breedings will serve as recipients. Saphenous artery segments will be isolated, mounted in the assay chamber for myogenic tone, and imaged intact by confocal microscopy as in Aim 1 while also measuring myogenic tone. By coupling the localization imaging with the vessel hanging assay, the presence or absence of MΦs can be correlated with tone changes.

In a complementary approach, an emigration blocking strategy is used to prevent MΦs from leaving the blood space. In the experiments, total cell isolates from the two heterozygous monocyte reporter mice (which have intact CX₃R1 and CCR2 receptors) will be used, and commercially available blocking antibodies targeting either CX₃R1 or CCR2 will be co-injected. These antibodies have been shown to minimize recruitment of monocytes/macrophages in non-inflamed and inflamed tissues, respectively (102, 103). In this way, the ability of macrophages/monocytes to leave the blood space after cell harvest can be reduced. This will complement the knockout experiments in which the absence of CX₃R1 or CCR2 may impact the cell composition of the adipose stromal populations, thereby skewing outcomes. This approach is particularly useful in experiments blocking macrophage/monocyte emigration in isolates prepared from SOD3 knockout mice. Performed identically as those described above, these SOD3-based experiments will assess superoxide dismutase activity centered in vessel wall-resident macrophages.

While not wishing to be bound by theory, it is suggested that macrophages/monocytes injected into the circulation enter into the vessel wall and that these vessel-resident cells are mediating vessel relaxation. In fact, MΦs routinely enter the vessel and are part of the normal adventitia cellular make up in arteries (29, 30). It is anticipated that an absence of myogenic relaxation with an absence of infiltrating MΦs (despite them being present in the animal circulation). The two different approaches, KO and antibody blocking, are intended to provide the same outcome by different but complimentary strategies. The blocking antibodies will probably not completely inhibit emigration. But, significant reductions have been routinely reported in the literature. Also, depletion experiments show that a significant effect on dilation even though removal of CD11b⁺ cells was not complete.

Throughout this document, various references are mentioned. All such references are incorporated herein by reference, including the references set forth in the following list:

### REFERENCES

1. Olsen T S, Larsen B, Herring M, Skriver E B, Lassen N A. Blood flow and vascular reactivity in collaterally perfused brain tissue. Evidence of an ischemic penumbra in patients with acute stroke. Stroke. 1983; 14 (3):332-41.
2. Jeremy R W, Hackworthy R A, Bautovich G, Hutton B F, Harris P J. Infarct artery perfusion and changes in left ventricular volume in the month after acute myocardial infarction. J Am Coll Cardiol. 1987;9 (5):989-95.
3. Faber J E, Zhang H, Lassance-Soares R M, Prabhakar P, Najafi A H, Burnett M S, et al. Aging causes collateral rarefaction and increased severity of ischemic injury in multiple tissues. Arteriosclerosis, thrombosis, and vascular biology. 2011;31 (8):1748-56. PMCID: 3141082.
4. Bauer S M, Bauer R J, Velazquez O C. Angiogenesis, vasculogenesis, and induction of healing in chronic wounds. Vasc Endovascular Surg. 2005;39 (4):293-306.
5. Georgi M K, Vigilance J, Dewar A M, Frame M D. Terminal arteriolar network structure/function and plasma cytokine levels in db/db and ob/ob mouse skeletal muscle. Microcirculation. 2011;18 (3):238-51. PMCID: 3081372.
6. Duncan H J, Faris I B. Skin vascular resistance and skin perfusion pressure as predictors of healing of ischemic lesion of the lower limb: influences of diabetes mellitus, hypertension, and age. Surgery. 1986;99 (4):432-8.
7. Futrakul P, Futrakul N, Sitprija V, Sensirivatana R, Watana D, Kingwatanakul P. Improved renal perfusion prevents disease progression in focal segmental glomerulosclerosis. Nephron. 1995;69 (3):351.
8. Claxton S, Fruttiger M. Oxygen modifies artery differentiation and network morphogenesis in the retinal vasculature. Dev Dyn. 2005;233 (3):822-8.
9. Darland D C, Massingham L J, Smith S R, Piek E, Saint-Geniez M, D'Amore P A. Pericyte production of cell-associated VEGF is differentiation-dependent and is associated with endothelial survival. DevBiol. 2003;264 (1):275-88.
10. Benjamin L E, Hemo I, Keshet E. A plasticity window for blood vessel remodelling is defined by pericyte coverage of the preformed endothelial network and is regulated by PDGF-B and VEGF. Development. 1998;125 (9):1591-8.
11. Hallerstam S, Larsson P T, Zuber E, Rosfors S. Carotid atherosclerosis is correlated with extent and severity of coronary artery disease evaluated by myocardial perfusion scintigraphy. Angiology. 2004;55 (3):281-8.
12. Cutolo M, Ferrone C, Pizzorni C, Soldano S, Seriolo B, Sulli A. Peripheral blood perfusion correlates with microvascular abnormalities in systemic sclerosis: a laser-Doppler and nailfold videocapillaroscopy study. J Rheumatol. 2010;37 (6):1174-80.
13. Pries A R, Hopfner M, le Noble F, Dewhirst M W, Secomb T W. The shunt problem: control of functional shunting in normal and tumour vasculature. Nat Rev Cancer. 2010;10 (8):587-93.
14. Pries A R, Cornelissen A J, Sloot A A, Hinkeldey M, Dreher M R, Hopfner M, et al. Structural adaptation and heterogeneity of normal and tumor microvascular networks. PLoS Comput Biol. 2009;5 (5):e1000394. PMCID: 2682204.
15. Humar R, Zimmerli L, Battegay E. Angiogenesis and hypertension: an update. J HumHypertens. 2009;23 (12):773-82.
16. Mourad J J, Levy B I. Mechanisms of antiangiogenic-induced arterial hypertension. Curr Hypertens Rep. 2011;13 (4):289-93.
17. Dharmashankar K, Widlansky M E. Vascular endothelial function and hypertension: insights and directions. Curr Hypertens Rep. 2010;12 (6):448-55. PMCID: 2982873.
18. de Wit C, Jahrbeck B, Schafer C, Bolz S S, Pohl U. Nitric oxide opposes myogenic pressure responses predominantly in large arterioles in vivo. Hypertension. 1998;31 (3):787-94.
19. Gupta S, Wright H M. Nebivolol: a highly selective beta 1-adrenergic receptor blocker that causes vasodilation by increasing nitric oxide. Cardiovasc Ther. 2008;26 (3):189-202.
20. Cecchi F, Sgalambro A, Baldi M, Sotgia B, Antoniucci D, Camici P G, et al. Microvascular dysfunction, myocardial ischemia, and progression to heart failure in patients with hypertrophic cardiomyopathy. Journal of cardiovascular translational research. 2009;2 (4):452-61.
21. Ruiz-Hurtado G, Delgado C. Nitric oxide pathway in hypertrophied heart: new therapeutic uses of nitric oxide donors. Journal of hypertension. 2010;28 Suppl 1:S56-61.
22. Herrick A L. Contemporary management of Raynaud's phenomenon and digital ischaemic complications. Curr Opin Rheumatol. 2011;23 (6):555-61.
23. Lopes L V, Sebastiao A M, Ribeiro J A. Adenosine and related drugs in brain diseases: present and future in clinical trials. Current topics in medicinal chemistry. 2011;11 (8):1087-101.
24. Piaggesi A, Vallini V, Iacopi E, Tedeschi A, Scatena A, Goretti C, et al. Iloprost in the management of peripheral arterial disease in patients with diabetes mellitus. Minerva cardioangiologica. 2011;59 (1):101-8.
25. Qi M, Hang C, Zhu L, Shi J. Involvement of endothelial-derived relaxing factors in the regulation of cerebral blood flow. Neurological sciences : official journal of the Italian Neurological Society and of the Italian Society of Clinical Neurophysiology. 2011;32(4):551-7.
26. Allen J D, Giordano T, Kevil C G. Nitrite and nitric oxide metabolism in peripheral artery disease. Nitric oxide : biology and chemistry/official journal of the Nitric Oxide Society. 2012;26 (4):217-22. PMCID: PMC3360821.
27. Gori T, Fineschi M. Two coronary “orphan” diseases in search of clinical consideration: coronary syndromes x and y. Cardiovasc Ther. 2012;30 (2):e58-65.
28. Toblli J E, DiGennaro F, Giani J F, Dominici F P. Nebivolol: impact on cardiac and endothelial function and clinical utility. Vascular health and risk management. 2012;8:151-60. PMCID: PMC3310359.
29. Majesky M W, Dong X R, Hoglund V, Daum G, Mahoney W M, Jr. The adventitia: a progenitor cell niche for the vessel wall. Cells Tissues Organs. 2012;195 (1-2):73-81. PMCID: PMC3257816.
30. Majesky M W, Dong X R, Hoglund V, Mahoney W M, Jr., Daum G. The adventitia: a dynamic interface containing resident progenitor cells. Arterioscler Thromb Vasc Biol. 2011;31 (7):1530-9. PMCID: PMC3382115.
31. Hong S J, Traktuev D O, March K L. Therapeutic potential of adipose-derived stem cells in vascular growth and tissue repair. CurrOpinOrgan Transplant. 2010;15 (1):86-91.
32. Gimble J M, Guilak F, Bunnell B A. Clinical and preclinical translation of cell-based therapies using adipose tissue-derived cells. Stem Cell ResTher. 2010;1 (2):19.
33. Gimble J M, Katz A J, Bunnell B A. Adipose-derived stem cells for regenerative medicine. Circ Res. 2007;100 (9):1249-60.
34. Yi T, Song S U. Immunomodulatory properties of mesenchymal stem cells and their therapeutic applications. Arch Pharm Res. 2012;35 (2):213-21.
35. McIntosh K R. Evaluation of cellular and humoral immune responses to allogeneic adipose-derived stem/stromal cells. Methods Mol Biol. 2011;702:133-50.
36. Darlington P J, Boivin M N, Bar-Or A. Harnessing the therapeutic potential of mesenchymal stem cells in multiple sclerosis. Expert Rev Neurother. 2011;11 (9):1295-303. PMCID: 3234364.
37. Pachon-Pena G, Yu G, Tucker A, Wu X, Vendrell J, Bunnell B, et al. Stromal stem cells from adipose tissue and bone marrow of age matched female donors display distinct immunophenotypic profiles. J Cell Physiol. 2010.
38. Lolmede K, Duffaut C, Zakaroff-Girard A, Bouloumie A. Immune cells in adipose tissue: key players in metabolic disorders. Diabetes Metab. 2011;37 (4):283-90.
39. Park H K, Cho K S, Park H Y, Shin D H, Kim Y K, Jung J S, et al. Adipose-derived stromal cells inhibit allergic airway inflammation in mice. Stem Cells Dev. 2010;19 (11):1811-8.
40. Lumeng C N, Deyoung S M, Bodzin J L, Saltiel A R. Increased inflammatory properties of adipose tissue macrophages recruited during diet-induced obesity. Diabetes. 2007;56 (1):16-23.
41. Pollard J W. Trophic macrophages in development and disease. Nat Rev Immunol. 2009;9 (4):259-70.
42. Nunes S S, Maijub J G, Krishnan L, Ramakrishnan V M, Clayton L R, Williams S K, et al. Generation of a functional liver tissue mimic using adipose stromal vascular fraction cell-derived vasculatures. Scientific reports. 2013;3:2141. PMCID: PMC3701895.
43. Chang C C, Krishnan L, Nunes S S, Church K H, Edgar L T, Boland E D, et al. Determinants of microvascular network topologies in implanted neovasculatures. Arterioscler Thromb Vasc Biol. 2012;32 (1):5-14.
44. Leblanc A J, Touroo J S, Hoying J B, Williams S K. Adipose stromal vascular fraction cell construct sustains coronary microvascular function after acute myocardial infarction. Am J Physiol Heart Circ Physiol. 2012;302 (4):H973-82.
45. Leblanc A J, Krishnan L, Sullivan C J, Williams S K, Hoying J B. Microvascular Repair: Post-Angiogenesis Vascular Dynamics. Microcirculation. 2012.
46. Cardinal T R, Struthers K R, Kesler T J, Yocum M D, Kurjiaka D T, Hoying J B. Chronic hindlimb ischemia impairs functional vasodilation and vascular reactivity in mouse feed arteries. Frontiers in Physiology. 2011;2.
47. Nunes S S, Rekapally H, Chang C C, Hoying J B. Vessel arterial-venous plasticity in adult neovascularization. PLoS One. 2011;6 (11):e27332. PMCID: 3221655.
48. Nunes S S, Greer K A, Stiening C M, Chen H Y, Kidd K R, Schwartz M A, et al. Implanted microvessels progress through distinct neovascularization phenotypes. MicrovascRes. 2010;79 (1):10-20.
49. Nunes S S, Krishnan L, Gerard C S, Dale J R, Maddie M A, Benton R L, et al. Angiogenic Potential of Microvessel Fragments is Independent of the Tissue of Origin and can be Influenced by the Cellular Composition of the Implants. Microcirculation. 2010;17 (7):557-67. PMCID: PMC3057771.
50. Cardinal T R, Hoying J B. A modified fluorescent microsphere-based approach for determining resting and hyperemic blood flows in individual murine skeletal muscles. VasculPharmacol. 2007;47 (1):48-56.
51. Shepherd B R, Hoying J B, Williams S K. Microvascular transplantation after acute myocardial infarction. Tissue Eng. 2007;13 (12):2871-9.
52. Gruionu G, Hoying J B, Gruionu L G, Laughlin M H, Secomb T W. Structural adaptation increases predicted perfusion capacity after vessel obstruction in arteriolar arcade network of pig skeletal muscle. AmJPhysiol Heart CircPhysiol. 2005;288 (6):H2778-H84.
53. Gruionu G, Hoying J B, Pries A R, Secomb T W. Structural remodeling of mouse gracilis artery after chronic alteration in blood supply. AmJPhysiol Heart CircPhysiol. 2005;288 (5):H2047-H54.
54. Shepherd B R, Chen H Y, Smith C M, Gruionu G, Williams S K, Hoying J B. Rapid perfusion and network remodeling in a microvascular construct after implantation. ArteriosclerThrombVascBiol. 2004;24 (5):898-904.
55. Sullivan C J, Doetschman T, Hoying J B. Targeted disruption of the Fgf2 gene does not affect vascular growth in the mouse ischemic hindlimb. JApplPhysiol. 2002;93 (6):2009-17.
56. Dimayuga P C, Li H, Chyu K Y, Fredrikson G N, Nilsson J, Fishbein M C, et al. T cell modulation of intimal thickening after vascular injury: the bimodal role of IFN-gamma in immune deficiency. Arterioscler Thromb Vasc Biol. 2005;25 (12):2528-34.
57. Dimayuga P, Cercek B, Oguchi S, Fredrikson G N, Yano J, Shah P K, et al Inhibitory effect on arterial injury-induced neointimal formation by adoptive B-cell transfer in Rag-1 knockout mice. Arterioscler Thromb Vasc Biol. 2002;22 (4):644-9.
58. Murray P J, Wynn T A. Protective and pathogenic functions of macrophage subsets. Nat Rev Immunol. 2011;11 (11):723-37. PMCID: PMC3422549.
59. Morris D L, Singer K, Lumeng C N. Adipose tissue macrophages: phenotypic plasticity and diversity in lean and obese states. Current opinion in clinical nutrition and metabolic care. 2011;14 (4):341-6.
60. LeBlanc A J, Moseley A M, Chen B T, Frazer D, Castranova V, Nurkiewicz T R. Nanoparticle inhalation impairs coronary microvascular reactivity via a local reactive oxygen species-dependent mechanism. Cardiovascular toxicology. 2010;10 (1):27-36. PMCID: PMC2825710.
61. Lesniewski L A, Connell M L, Durrant J R, Folian B J, Anderson M C, Donato A J, et al. B6D2F1 Mice are a suitable model of oxidative stress-mediated impaired endothelium-dependent dilation with aging. The journals of gerontology Series A, Biological sciences and medical sciences. 2009;64 (1):9-20. PMCID: PMC2691198.
62. Faraci F M, Didion S P. Vascular protection: superoxide dismutase isoforms in the vessel wall. Arterioscler Thromb Vasc Biol. 2004;24 (8):1367-73.
63. Schubert R, Lidington D, Bolz S S. The emerging role of Ca2+ sensitivity regulation in promoting myogenic vasoconstriction. Cardiovasc Res. 2008;77 (1):8-18.
64. Wagenfeld L, von Domarus F, Weiss S, Klemm M, Richard G, Zeitz O. The effect of reactive oxygen species on the myogenic tone of rat ophthalmic arteries with and without endothelium. Graefe's archive for clinical and experimental ophthalmology=Albrecht von Graefes Archiv fur klinische and experimentelle Ophthalmologie. 2013.
65. Lai E Y, Wellstein A, Welch W J, Wilcox C S. Superoxide modulates myogenic contractions of mouse afferent arterioles. Hypertension. 2011;58 (4):650-6. PMCID: PMC3190586.
66. Matoba T, Shimokawa H, Nakashima M, Hirakawa Y, Mukai Y, Hirano K, et al. Hydrogen peroxide is an endothelium-derived hyperpolarizing factor in mice. J Clin Invest. 2000;106 (12):1521-30. PMCID: PMC387255.
67. Kang L S, Reyes R A, Muller-Delp J M. Aging impairs flow-induced dilation in coronary arterioles: role of NO and H(2)O(2). Am J Physiol Heart Circ Physiol. 2009;297 (3):H1087-95. PMCID: PMC2755981.
68. Manni M L, Tomai L P, Norris C A, Thomas L M, Kelley E E, Salter R D, et al. Extracellular superoxide dismutase in macrophages augments bacterial killing by promoting phagocytosis. Am J Pathol. 2011;178 (6):2752-9. PMCID: PMC3124355.
69. Auwerx J H, Chait A, Wolfbauer G, Deeb S S. Loss of copper-zinc superoxide dismutase gene expression in differentiated cells of myclo-monocytic origin. Blood. 1989;74 (5):1807-10.
70. Phillips S A, Hatoum O A, Gutterman D D. The mechanism of flow-induced dilation in human adipose arterioles involves hydrogen peroxide during CAD. Am J Physiol Heart Circ Physiol. 2007;292 (1):H93-100.
71. Liesveld J L, Winslow J M, Frediani K E, Ryan D H, Abboud C N. Expression of integrins and examination of their adhesive function in normal and leukemic hematopoietic cells. Blood. 1993;81 (1):112-21.
72. Stoneman V, Braganza D, Figg N, Mercer J, Lang R, Goddard M, et al. Monocyte/macrophage suppression in CD11b diphtheria toxin receptor transgenic mice differentially affects atherogenesis and established plaques. Circ Res. 2007;100 (6):884-93. PMCID: 2040259.
73. Brancato S K, Albina J E. Wound macrophages as key regulators of repair: origin, phenotype, and function. Am J Pathol. 2011;178 (1):19-25. PMCID: 3069845.
74. Motoike T, Loughna S, Perens E, Roman BL, Liao W, Chau T C, et al. Universal GFP reporter for the study of vascular development. Genesis. 2000;28 (2):75-81.
75. Cailhier J F, Partolina M, Vuthoori S, Wu S, Ko K, Watson S, et al. Conditional macrophage ablation demonstrates that resident macrophages initiate acute peritoneal inflammation. Journal of immunology (Baltimore, Md.: 1950). 2005;174 (4):2336-42.
76. Altmann C, Andres-Hernando A, McMahan R H, Ahuja N, He Z, Rivard C J, et al. Macrophages mediate lung inflammation in a mouse model of ischemic acute kidney injury. Am J Physiol Renal Physiol. 2011.
77. Mosser D M. The many faces of macrophage activation. JLeukocBiol. 2003;73 (2):209-12.
78. Clifford P S, Ella S R, Stupica A J, Nourian Z, Li M, Martinez-Lemus L A, et al. Spatial distribution and mechanical function of elastin in resistance arteries: a role in bearing longitudinal stress. Arterioscler Thromb Vasc Biol. 2011;31 (12):2889-96. PMCID: PMC3380608.
79. Koh Y J, Koh B I, Kim H, Joo H J, Jin H K, Jeon J, et al. Stromal vascular fraction from adipose tissue forms profound vascular network through the dynamic reassembly of blood endothelial cells. Arterioscler Thromb Vasc Biol. 2011;31 (5):1141-50.
80. Zimmerlin L, Donnenberg V S, Pfeifer M E, Meyer E M, Peault B, Rubin J P, et al. Stromal vascular progenitors in adult human adipose tissue. Cytometry A. 2010;77 (1):22-30.
81. Leobon B, Roncalli J, Joffre C, Mazo M, Boisson M, Barreau C, et al. Adipose-derived cardiomyogenic cells: in vitro expansion and functional improvement in a mouse model of myocardial infarction. CardiovascRes. 2009;83 (4):757-67.
82. Yoshimura K, Sato K, Aoi N, Kurita M, Hirohi T, Harii K. Cell-assisted lipotransfer for cosmetic breast augmentation: supportive use of adipose-derived stem/stromal cells. Aesthetic PlastSurg. 2008;32 (1):48-55.
83. Mitchell J B, McIntosh K, Zvonic S, Garrett S, Floyd Z E, Kloster A, et al. Immunophenotype of human adipose-derived cells: temporal changes in stromal-associated and stem cell-associated markers. Stem Cells. 2006;24 (2):376-85.
84. Melief S M, Zwaginga J J, Fibbe W E, Roelofs H. Adipose Tissue-Derived Multipotent Stromal Cells Have a Higher Immunomodulatory Capacity Than Their Bone Marrow-Derived Counterparts. Stem Cells Transl Med. 2013.
85. Schweitzer K S, Johnstone B H, Garrison J, Rush N I, Cooper S, Traktuev D O, et al. Adipose stem cell treatment in mice attenuates lung and systemic injury induced by cigarette smoking Am J Respir Crit Care Med. 2011;183 (2):215-25. PMCID: 3040390.
86. Casteilla L, Planat-Benard V, Laharrague P, Cousin B. Adipose-derived stromal cells: Their identity and uses in clinical trials, an update. World journal of stem cells. 2011;3 (4):25-33. PMCID: PMC3097937.
87. Koh T J, DiPietro L A. Inflammation and wound healing: the role of the macrophage. Expert Rev Mol Med. 2011;13:e23.
88. Driscoll K E. Macrophage inflammatory proteins: biology and role in pulmonary inflammation. ExpLung Res. 1994;20 (6):473-90.
89. Kang L S, Chen B, Reyes R A, Leblanc A J, Teng B, Mustafa S J, et al. Aging and estrogen alter endothelial reactivity to reactive oxygen species in coronary arterioles. Am J Physiol Heart Circ Physiol. 2011;300 (6):H2105-15. PMCID: PMC3119103.
90. Laurindo F R, Pedro Mde A, Barbeiro H V, Pileggi F, Carvalho M H, Augusto O, et al. Vascular free radical release. Ex vivo and in vivo evidence for a flow-dependent endothelial mechanism. Circ Res. 1994;74 (4):700-9.
91. Benov L, Sztejnberg L, Fridovich I. Critical evaluation of the use of hydroethidine as a measure of superoxide anion radical. Free radical biology & medicine. 1998;25 (7):826-31
92. Oury T D, Ho Y S, Piantadosi C A, Crapo J D. Extracellular superoxide dismutase, nitric oxide, and central nervous system O2 toxicity. Proc Natl Acad Sci USA. 1992;89 (20):9715-9. PMCID: PMC50203.
93. Heikkila R E, Cabbat F S, Cohen G. In vivo inhibition of superoxide dismutase in mice by diethyldithiocarbamate. J Biol Chem. 1976;251 (7):2182-5.
94. Lee M R, Li L, Kitazawa T. Cyclic GMP causes Ca2+ desensitization in vascular smooth muscle by activating the myosin light chain phosphatase. J Biol Chem. 1997;272 (8):5063-8.
95. Gongora M C, Lob H E, Landmesser U, Guzik T J, Martin W D, Ozumi K, et al. Loss of extracellular superoxide dismutase leads to acute lung damage in the presence of ambient air: a potential mechanism underlying adult respiratory distress syndrome. Am J Pathol. 2008;173 (4):915-26. PMCID: PMC2543061.
96. Carlsson L M, Jonsson J, Edlund T, Marklund S L. Mice lacking extracellular superoxide dismutase are more sensitive to hyperoxia. Proc Natl Acad Sci USA. 1995;92 (14):6264-8. PMCID: PMC41498.
97. Boillee S, Yamanaka K, Lobsiger C S, Copeland N G, Jenkins N A, Kassiotis G, et al. Onset and progression in inherited ALS determined by motor neurons and microglia. Science. 2006;312 (5778):1389-92.
98. Shi C, Pamer E G. Monocyte recruitment during infection and inflammation. Nat Rev Immunol. 2011;11 (11):762-74.
99. Kuziel W A, Morgan S J, Dawson T C, Griffin S, Smithies O, Ley K, et al. Severe reduction in leukocyte adhesion and monocyte extravasation in mice deficient in CC chemokine receptor 2. Proc Natl Acad Sci USA. 1997;94 (22):12053-8. PMCID: PMC23699.
100. Auffray C, Fogg D, Garfa M, Elain G, Join-Lambert O, Kayal S, et al. Monitoring of blood vessels and tissues by a population of monocytes with patrolling behavior. Science. 2007;317 (5838):666-70.
101. Cochain C, Rodero M P, Vilar J, Recalde A, Richart A L, Loinard C, et al. Regulation of monocyte subset systemic levels by distinct chemokine receptors controls post-ischaemic neovascularization. Cardiovasc Res. 2010;88 (1):186-95.
102. Geissmann F, Jung S, Littman D R. Blood monocytes consist of two principal subsets with distinct migratory properties. Immunity. 2003;19 (1):71-82.
103. Oh D J, Dursun B, He Z, Lu L, Hoke T S, Ljubanovic D, et al. Fractalkine receptor (CX3CR1) inhibition is protective against ischemic acute renal failure in mice. Am J Physiol Renal Physiol. 2008;294 (1):F264-71.
104. Cao, Y. A., et al. Shifting foci of hematopoiesis during reconstitution from single stem cells. Proc Natl Acad Sci USA 101, 221-226 (2004).
105. Motoike, T., et al. Universal GFP reporter for the study of vascular development. Genesis 28, 75-81 (2000).
106. Moroi, M., et al. Interaction of genetic deficiency of endothelial nitric oxide, gender, and pregnancy in vascular response to injury in mice. J Clin Invest 101, 1225-1232 (1998).
107. Kang, L. S., et al. Aging and estrogen alter endothelial reactivity to reactive oxygen species in coronary arterioles. Am J Physiol Heart Circ Physiol 300, H2105-2115 (2011).
108. LeBlanc, A. J., et al. Nanoparticle inhalation impairs coronary microvascular reactivity via a local reactive oxygen species-dependent mechanism. Cardiovascular toxicology 10, 27-36 (2010).
109. Phillips, S. A., Hatoum, O. A. & Gutterman, D. D. The mechanism of flow-induced dilation in human adipose arterioles involves hydrogen peroxide during CAD. Am J Physiol Heart Circ Physiol 292, H93-100 (2007).
110. Clifford, P. S., et al. Spatial distribution and mechanical function of elastin in resistance arteries: a role in bearing longitudinal stress. Arterioscler Thromb Vasc Biol 31, 2889-2896 (2011).
111. Wu, W. & Chaer, R. A. Nonarteriosclerotic vascular disease. The Surgical clinics of North America 93, 833-875, viii (2013).
112. Christensen, J. E., Andreasen, S. O., Christensen, J. P. & Thomsen, A. R. CD11b expression as a marker to distinguish between recently activated effector CD8(+) T cells and memory cells. International immunology 13, 593-600 (2001).
113. Maddox, D. E., Shibata, S. & Goldstein, I. J. Stimulated macrophages express a new glycoprotein receptor reactive with Griffonia simplicifolia I-B4 isolectin. Proc Natl Acad Sci USA 79, 166-170 (1982).
114. Laitinen, L. Griffonia simplicifolia lectins bind specifically to endothelial cells and some epithelial cells in mouse tissues. The Histochemical journal 19, 225-234 (1987).
115. Hill, M. A., Meininger, G. A., Davis, M. J. & Laher, I. Therapeutic potential of pharmacologically targeting arteriolar myogenic tone. Trends Pharmacol Sci 30, 363-374 (2009).
116. Palomares, S. M. & Cipolla, M. J. Myogenic Tone as a Therapeutic Target for Ischemic Stroke. Curr Vasc Pharmacol (2013).
117. Fukai, T., Folz, R. J., Landmesser, U. & Harrison, D. G. Extracellular superoxide dismutase and cardiovascular disease. Cardiovasc Res 55, 239-249 (2002).
118. Lumeng, C. N., DelProposto, J. B., Westcott, D. J. & Saltiel, A. R. Phenotypic switching of adipose tissue macrophages with obesity is generated by spatiotemporal differences in macrophage subtypes. Diabetes 57, 3239-3246 (2008).
119. Dong, Z. M., Gutierrez-Ramos, J. C., Coxon, A., Mayadas, T. N. & Wagner, D. D. A new class of obesity genes encodes leukocyte adhesion receptors. Proc Natl Acad Sci USA 94, 7526-7530 (1997)
120. Morris, D. L., et al. Adipose tissue macrophages function as antigen-presenting cells and regulate adipose tissue CD4+ T cells in mice. Diabetes 62, 2762-2772 (2013).
121. Michael R. Phillips, MDa, Hiroki Yamaguchi, MDa, Virginia M. Miller, PhDb, Stuart Williams, PhDc, James J. Morris, MDa, Hartzell V. Schaff, MDa Endothelial sodding of the permaflow prosthetic coronary artery bypass conduit. Ann Thorac Surg 1998; 66:1191-1197.

One of ordinary skill in the art will recognize that additional embodiments and implementations are also possible without departing from the teaching of the present disclosure or the scope of the claims which follow. This description and, particularly, the specific details of the exemplary implementation disclosed is given primarily for clarity of understanding and no unnecessary limitations are to be understood therefrom, for modifications will be apparent and obvious to one of ordinary skill in the art upon reading this disclosure and may be made without departing from the spirit or scope of the present disclosure. Furthermore, the foregoing description is for the purpose of illustration only, and not for the purpose of limitation.

### INCORPORATION BY REFERENCE

All publications, patents, and patent applications mentioned in this description are herein incorporated by reference to the same extent as if each individual publication, patent, or patent application was specifically and individually indicated to be incorporated by reference.

What is claimed is:

1. A method of treating a blood vessel disease, comprising administering to a subject a composition comprising a stromal vascular fraction cell population.

2. The method of claim 1, wherein administering the composition to the subject comprises intravenously injecting the composition.

3. The method of claim 1, further comprising the step of isolating the stromal vascular fraction cell population from adipose tissue of the subject prior to administering the composition to the subject.

4. The method of claim 1, wherein administering the stromal vascular fraction cell population to the subject comprises distributing the stromal vascular fraction cell population in at least one of the intima, media, and adventitia of a blood vessel of the subject.

5. The method of claim 1, wherein administering the stromal vascular fraction cell population to the subject increases an amount of a vasodilatory agent in the subject.

6. The method of claim 1, wherein administering the composition increases the activity of a vasodilatory agent in the intima media and adventitia of a blood vessel of the subject.

7. The method of claim 5, wherein the vasodilatory agent is chosen from nitric oxide, histamine, prostacyclin, prostaglandin E2, prostaglandin I2, leukotriene C4, leukotriene D4, leukotriene E4, vasoactive intestinal peptide (VIP), adenosine, adenosine triphosphate, adenosine diphosphate, L-arginine, bradykinin, substance P, nicotinic acid, platelet activating factor, carbon dioxide, lactic acid, natriuretic peptide, heparin, heparin sulfate, and endothelium derived hyperpolarizing factor.

8. The method of claim 6, wherein the vasodilatory agent is selected from the group consisting of nitric oxide, histamine, prostacyclin, prostaglandin E2, prostaglandin I2, leukotriene C4, leukotriene D4, leukotriene E4, vasoactive intestinal peptide (VIP), adenosine, adenosine triphosphate, adenosine diphosphate, L-arginine, bradykinin, substance P, nicotinic acid, platelet activating factor, carbon dioxide, lactic acid, natriuretic peptide, heparin, heparin sulfate, and endothelium derived hyperpolarizing factor.

9. The method of claim 1, wherein administering the composition to the subject decreases an amount of vasoconstriction in a blood vessel of the subject.

10. The method of claim 1, wherein administering the composition decreases the activity of a vasoconstricting agent in a blood vessel of a subject.

11. The method of claim 10, wherein the vasoconstricting agent is selected from the group consisting of prostaglandin F2, thromboxane A2, and thromboxane B2.

12. The method of claim 1, wherein administering the stromal vascular fraction cell population to the subject comprises distributing the stromal vascular fraction cell population in the bone marrow of the subject.

13. The method of claim 14, wherein distributing the stromal vascular fraction cell population in the bone marrow of the subject increases an amount of red blood cells, white blood cells, megakaryocytes, platelets or a combination thereof in the subject.

14. A composition, comprising a population of stromal vascular fraction cells, the population of stromal vascular fraction cells including one or more macrophages.

15. The composition of claim 14, wherein the composition is formulated for intravenous injection.

16. The composition of claim 15, further comprising a pharmaceutically-acceptable carrier.

17. A kit, comprising a container including a population of stromal vascular fraction cells.

18. The kit of claim 17, further comprising a syringe for injecting the population of stromal vascular fraction cells.

19. The kit of claim 17, wherein the population of stromal vascular fraction cells has been depleted of macrophages.

20. The kit of claim 17, wherein the population of stromal vascular fraction cells has been enriched with macrophages isolated from a stromal vascular fraction of adipose tissue.
